(19) United States
(12) Patent Application Publication
(10) Pub. No.: US 2023/0047313 A1
(43) Pub. Date: Feb. 16, 2023

(54) TREATING HEART DISEASE IN MUSCULAR DYSTROPHY PATIENTS

(71) Applicant: Children`s Medical Center Corporation, Boston, MA (US)

(72) Inventors: Da-Zhi Wang, Newton, MA (US); Zhanpeng Huang, Las Vegas, NV (US); Jianming Liu, Boston, MA (US)

(21) Appl. No.: 17/786,154

(22) PCT Filed: Dec. 16, 2020
(86) PCT No.: PCT/US2020/065348

§ 371 Date: Jun. 16, 2022

Related U.S. Application Data
(60) Provisional application No. 62/948,521, filed on Dec. 16, 2019.

(51) Int. Cl. A61K 31/437; A61P 9/04

(52) U.S. Cl. CPC A61K 31/437; A61P 9/04

(57) ABSTRACT

Methods of treating or reducing risk of developing cardiomyopathy or heart failure in a subject, comprising administering to a subject in need thereof a therapeutically effective amount of an inhibitor of NADPH oxidase 4 (Nox4).

### CLAIM OF PRIORITY

This application claims priority under 35 USC § 119(e) to U.S. Patent Application Ser. No. 62/948,521, filed on Dec. 16, 2019. The entire contents of the foregoing are hereby incorporated by reference.

### STATEMENT REGARDING FEDERAL FUNDING

This invention was made with government support under Grant Nos. HL116919 and HL149401 awarded by the National Institutes of Health. The government has certain rights in the invention.

### FIELD OF THE INVENTION

This invention relates to methods of treating or reducing risk of developing cardiomyopathy or heart failure in a subject, comprising administering to a subject in need thereof a therapeutically effective amount of an inhibitor of NADPH oxidase 4 (Nox4).

### BACKGROUND

Heart failure (HF) is general believed to be the clinical consequence of deleterious cardiac remodeling. Pressure overload of the left ventricle can be induced by various clinical conditions, such as hypertension and aortic stenosis, which trigger pathological cardiac remodeling (Cohn et al., Journal of the American College of Cardiology. 2000; 35:569-582). Indeed, clinical data has revealed that hypertension is a major risk factor in the development of HF (Messerli et al., JACC. Heart failure. 2017; 5:543-551). Pathological changes in cardiac cells, including hypertrophic growth of cardiomyocytes, necrosis and apoptosis of cardiomyocytes, and activation of cardiac fibroblasts, are closely related to cardiac remodeling (Hill and Olson, The New England journal of medicine. 2008; 358:1370-138; Ahmad et al., Annual review of genomics and human genetics. 2005; 6:185-216). Complicated gene regulation events are thought to be involved in these processes (Frey and Olson, Annual review of physiology. 2003; 65:45-79). Since cardiac remodeling is now recognized as a determinant of HF, slowing or reversing remodeling has become a primary goal of HF therapy (Cohn et al., 2000). However, fully understanding the underlying molecular mechanisms is a prerequisite for developing new therapeutic approaches. Although several important signaling pathways, such as IGF (Welch et al., Circulation research. 2002; 90:641-648; Ren et al., Journal of molecular and cellular cardiology. 1999; 31:2049-2061), TGF-beta (Schultz et al., The Journal of clinical investigation. 2002; 109:787-796; Koitabashi et al., The Journal of clinical investigation. 2011; 121:2301-2312), Mitogen-activated protein kinases (Heineke and Molkentin, Nature reviews. Molecular cell biology. 2006; 7:589-600; Wang, Circulation. 2007; 116:1413-142), Calmodulin-Calcineurin signaling (Frey et al., Nature medicine. 2000; 6:1221-1227; Molkentin et al., Cell. 1998; 93:215-228), have been identified and studied in cardiac remodeling, the precise molecular mechanism of disease progression toward HF remains elusive. Identification of novel factors that regulate this transition will not only offer new entry points to better understand the responsible gene regulatory networks, but will also provide potential targets for therapeutic application.

Duchenne Muscular Dystrophy (DMD) is a genetic disorder caused by mutations in the X-linked dystrophin gene that affects the structure and function of striated muscle (cardiac and skeletal muscle) (Hoffman et al., Cell. 1987; 51:919-28; Monaco et al., Nature. 1986; 323:646-50; Chamberlain and Chamberlain Molecular therapy: the journal of the American Society of Gene Therapy. 2017; 25:1125-1131; Guiraud and Davies, Curr Opin Pharmacol. 2017; 34:36-48.). These mutations lead to muscle wasting and regeneration defects in skeletal muscle. Using genome editing technology, recent studies show that germline or somatic correction of the dystrophin mutation can restore muscle function (Long et al., Science. 2016; 351:400-3; Long et al., Science. 2014; 345:1184-1188; Min et al., Annu Rev Med. 2019; 70:239-255; Amoasii et al., Science. 2018; 362:86-91). However, much less is known about dystrophic defects in the heart, or dystrophic cardiomyopathy, which has become a leading cause of fatalities in DMD (Kamdar and Garry, J Am Coll Cardiol. 2016; 67:2533-46; McNally, Annu Rev Med. 2007; 58:75-88). Most importantly, the pathophysiological mechanisms involved in cardiac myocytes appear to differ significantly from those in skeletal myofibers (McNally, 2007). It has been suggested that mutations in dystrophy-causing genes may result in structural defects in myofibrils, leading to an increase in the permeability of myocytes. It is also suggested that mutations in muscle cells might trigger an inflammatory response, resulting in loss of myocytes and a decrease of muscle contractility. However, it remains unknown how these genetic mutations cause defects in the heart, resulting in dystrophic cardiomyopathy.

### SUMMARY

Provided herein are methods of treating subjects with inhibitors of NADPH oxidase 4 (Nox4).

Thus, provided herein are methods of treating cardiomyopathy or heart failure in a subject. Also provided herein are methods for reducing risk of development of cardiomyopathy or heart failure in a subject. Also provided are inhibitors of NADPH oxidase 4 (Nox4) for use in a method of treating cardiomyopathy or heart failure in a subject, or for reducing risk of development of cardiomyopathy or heart failure in a subject. Also provided herein are methods for treating a subject who has a muscular dystrophy, and inhibitors of NADPH oxidase 4 (Nox4) for use in a method of treating a subject who has a muscular dystrophy, e.g., to reduce skeletal muscle dysfunction.

The methods include administering to a subject in need thereof a therapeutically effective amount of an inhibitor of NADPH oxidase 4 (Nox4).

In some embodiments, the inhibitor is administered daily. In some embodiments, the inhibitor is selected from the group consisting of GKT137831; GKT136901; GSK2795039; VAS2870; perhexiline; VAS3947; compound 87 (2-(2-chlorophenyl)-5-[(1-methylpyrazol-3-yl)methyl]-4-[[methyl(pyridin-3-ylmethyl)amino]methyl]-1H-pyrazolo[4,3-c]pyridine-3,6-dione); compound 7c (10-benzyl-2-(2-chlorophenyl)-7,8,9,11-tetrahydro-3H-pyrazolo[4,5]pyrido[5,6-a][1,4]diazepine-1,5-dione; Gaggini et al., Bioorg Med Chem. 2011 Dec. 1; 19(23):6989-99); APX-115; VAS2870; fulvene-5; grindelic acid; phenantridinones; fluvenazine; DPI; suramin; ebselen; perhexiline; perhenazine; fluphenazine; and tertiary sulfonylureas.

In some embodiments, the subject has a muscular dystrophy. In some embodiments, the subject has Duchenne muscular dystrophy (DMD), Becker muscular dystrophy (BMD), and X-linked dilated cardiomyopathy (XL-DCM).

In some embodiments, the subject has sinus tachycardia, atrial arrhythmias, including atrial fibrillation, atrial flutter, atrial tachycardias, and/or left ventricular dysfunction, e.g., left ventricular ejection fraction (LVEF)<35%.

In some embodiments, the subject has DMD and is less than 10-12 years of age.

In some embodiments, the subject has left ventricular (LV) strain defects or myocardial fibrosis, but does not have left ventricular dysfuction.

### BRIEF DESCRIPTION OF THE DRAWINGS

FIGS. 1A-H. Loss of CIP accelerates dystrophic cardiomyopathy in young mdx mice. (A) Left ventricular posterior wall thickness at end-diastole (LVPW; d), Left ventricular internal dimension at end-diastole (LVID; d), and (B) Fractional shortening (FS) of 3-month-old CIP-KO; mdx dKO mice and their control littermates. (C) Gross heart morphology and (D) H&E staining of 3-month-old CIP-KO; mdx dKO mice and their control littermates. Bars=1.5 mm. (E) Sirius Red/Fast Green staining of 3-month-old CIP-KO; mdx dKO and control hearts. The fibrotic area was quantified. Bars=1 mm. qRT-PCR detection of expression of (F) heart disease marker genes, (G) fibrosis marker genes and (H) genes related to cell death in 3-month-old CIP-KO; mdx dKO hearts and littermate controls. N=3-5 for each group. The significance between groups was tested with 1-way ANOVA with post-hoc Tukey's test. *P<0.05; **P<0.01.

FIGS. 2A-E. Cardiac overexpression of CIP suppresses dystrophic cardiomyopathy in old mdx mice. (A) Left ventricular posterior wall thickness at end-diastole (LVPW; d), left ventricular internal dimension at end-diastole (LVID; d), and fractional shortening (FS) of 1-year-old CIP-OE; mdx compound mice and their control littermates. (B) Gross heart morphology, H&E staining and Sirius Red/Fast Green staining of 1-year-old CIP-OE; mdx compound mice and their control littermates. Bars=1.5 mm. The fibrotic area was quantified. qRT-PCR detection of expression of (C) heart disease marker genes, (D) fibrosis marker genes and (E) genes related to oxidative stress and cell death in 6-month-old CIP-OE; mdx compound hearts and littermate controls. The significance between groups was tested with 1-way ANOVA with post-hoc Tukey's test. *P<0.05; **P<0.01.

FIGS. 3A-B. Molecular pathways of dystrophic cardiomyopathy. (A) Gene ontology analysis of 1,744 up-regulated (P<0.05) and 1,492 down-regulated (P<0.05) genes in 3-month-old CIP-KO; mdx dKO hearts. The GO terms are ranked by FDR values. (B) Gene ontology analysis of 356 up-regulated (P<0.05) and 435 down-regulated (P<0.05) genes in 1-year-old CIP-OE; mdx hearts. The GO terms are ranked by FDR values.

FIGS. 4A-H. Oxidative stress and Nox4 are key inducers of dystrophic cardiomyopathy. qRT-PCR detection of expression of (A) Nox4 and (B) other oxidative stress-related genes in 3-month-old CIP-KO; mdx dKO hearts and controls. N=3-5 for each group. (C) GSSG/GSH ratio in 3-month-old CIP-KO; mdx dKO hearts and controls. N=4 for each group. (D) qRT-PCR detection of the expression of oxidative stress-related genes in 6-month-old CIP-OE; mdx hearts and controls. N=4 for each group. (E) GSSG/GSH ratio in 1-year-old CIP-OE; mdx hearts and controls. N=3-4 for each group. (F) Left ventricular internal dimension at end-diastole (LVID; d), left ventricular internal dimension at end-systole (LVID; s), and fractional shortening (FS) of 2-month-old mdx and control mice injected with AAV-Nox4 or control virus. N of each group is indicated. (G) Sirius Red/Fast Green staining of heart sections of 2-month-old mdx and control mice injected with AAV-Nox4 or control virus. The fibrotic area was quantified. Bar=1 mm. (H) qRT-PCR detection of gene expression in the hearts of 2-month-old mdx and control mice injected with AAV-Nox4 or control virus. N=3-5 for each group. The significance between groups was tested with 1-way ANOVA with post-hoc Tukey's test. *P<0.05; **P<0.01.

FIGS. 5A-J. CIP regulates the CnA-NFAT-Nox4 pathway to regulate dystrophic cardiomyopathy. (A) Immunohistochemistry detecting CIP protein in wildtype (WT) and CIP knockout (KO) adult mouse heart sections (left panels). Bars=50 μm. Immunohistochemistry detecting CIP and dystrophin protein in adult mouse heart sections (right panels). Bar=20 μm. (B) Coimmunoprecipitation assays showing the interaction between Myc-tagged CIP and Flag-tagged C-terminal of dystrophin (DMD-C) or Flag-tagged Lmo7, but not Flag-tagged N-terminal of dystrophin (DMD-N). 5% cell lysate was used as input to demonstrate the expression of tagged proteins. (C) Coimmunoprecipitation assays showing the interaction between Myc-tagged CIP and Flag-tagged CnA, but not Flag-tagged CnB or Flag-tagged CIB1. 5% cell lysate was used as input to demonstrate the expression of tagged proteins. (D) Green florescence images of Cos-7 mammalian cells cotransfected with EGFP-tagged NFAT and indicated expression plasmids. Cells with and without nuclear dominant GFP signals are indicated with grey and white arrows, respectively. Nuclei were stained with Hoechst 33342. (E) Quantification of the percentage of GFP positive cells with nuclear dominant GFP signals. (F) CIP represses CnA-mediated and NFAT-mediated transactivation of the NFAT-luciferase reporter in a dose-dependent manner. Results are presented as relative luciferase activity, in which the control is assigned a value of 1. Data were generated from at least three independent experiments. (G) qRT-PCR detection of Nox4 gene expression in phenylephrine-treated neonatal rat cardiomyocytes when CIP was overexpressed or knocked down. (H) qRT-PCR detection of CIP and Nox4 expression in 4-week-old CnA-tg and control hearts injected with AAV-CIP or control virus. N=4-5 for each group. (I) GSSG/GSH ratio in 4-week-old CnA-tg and control hearts injected with AAV-CIP or control virus. N=4 for each group. The significance between groups was tested with 1-way ANOVA with post-hoc Tukey's test. *P<0.05; **P<0.01. (J) A proposed working model of CIP regulating oxidative stress in dystrophic cardiomyocytes.

FIGS. 6A-E. GATA4 regulates the expression of CIP. (A) Distribution of reported binding sequences for cardiac transcriptional factors around the transcriptional starting site of CIP gene in a genome browser. Arrows indicate GATA4 binding sequences. (B) The expression of CIP when GATA4 and/or Mef2a were knocked down in HL1 cells (converted from reported data⁵¹). (C) Luciferase reporter assay of reporters with full length or truncated CIP promotors with or without GATA4 activation. Grey bars on the left indicate potential GATA4 binding sites. (D) Conservation of potential GATA4 binding motifs in mammalian, which are shown in grey letters. Asterisks indicate the conserved nucleotides. Shown are SEQ ID NOs: 57-59 (mouse), 60-62 (human), 63-65 (dog), and 66-68 (elephant). (E) Luciferase reporter assay of reporters with full wildtype or mutant CIP promotors with or without GATA4 activation. Grey boxes on the left indicate potential GATA4 binding motifs. Boxes with cross indicate mutant motifs.

FIGS. 7A-D. CIP expression is down-regulated in diseased human hearts. (A) Relative gene expression indicated by log counts per million (CPM) from disease human heart RNA-seq data. N number for each group is show. The significance between each category of heart disease and control was tested with 1-way ANOVA with post-hoc Tukey's test and shown. (B) Western blotting detecting the expression of CIP in human hearts with dilated cardiomyopathy (DCM) and controls. GAPDH served as loading control. (C) The ranking of enriched KEGG pathways in the gene set having strong expression correlation coefficient with CIP, which is determined by Spearman correlation coefficient (SCC). SCC between CIP and individual genes genome-wide from 194 human hearts was calculated. Pathways were ranked by the adjusted P value. (D) The expression of CIP has strong expression correlation coefficient with oxidative phosphorylation (OXPHOS) related genes in human heart (p=6.77E10⁻¹³). OXPHOS genes with SCC>0.4 are shown in Circos plot. The color of line linked between CIP and each gene indicates the expression correlation coefficient. A darker line suggests a stronger expression correlation coefficient.

FIGS. 8A-D. Cardiac-overexpression of CIP preserves cardiac function during the disease progression toward heart failure. (A) Representative echocardiographic images from indicated group at 8 weeks after operation. (B) Left ventricular posterior wall thickness at end-diastole (LVPW; d), (C) Left ventricular internal dimension at end-diastole (LVID; d) and (D) Fractional shortening (FS) of CIP-OE mice and their control littermates with TAC or Sham operation determined by echocardiography at 2 weeks (2w) and 8 weeks (8w) after operation. N number for each group is show. The significance between groups was tested with 1-way ANOVA with post-hoc Tukey's test. *P<0.05; **P<0.01.

FIGS. 9A-E. CIP inhibits cardiac remodeling in the transition from cardiac hypertrophy to failure. (A) The ratio of ventricle weight vs. body weight of CIP-OE mice and their control littermates at 8 weeks after TAC or sham operation. N number of each group is shown. (B) Haematoxylin Eosin (H&E) staining of hearts from CIP-OE mice and their control littermates at 8 weeks after TAC or sham operation. Bars=1 mm. (C) Fast green and Sirius red staining of hearts from CIP-OE mice and their control littermates at 8 weeks after TAC or sham operation. The fibrotic area was quantified. Bars=1 mm. (D) Wheat germ agglutinin staining detecting the cross area of cardiomyocytes in TAC- or sham-operated CIP-OE hearts and littermate controls. The size of cardiomyocyte was quantified. Bars=40 μm. (E) qRT-PCR detection of expression of cardiac fibrosis and heart disease marker genes in TAC- or sham-operated CIP-OE hearts and littermate controls. N=4 for each group. The significance between groups was tested with 1-way ANOVA with post-hoc Tukey's test. *P<0.05; **P<0.01.

FIGS. 10A-E. IGF1R and mTORC2 signaling pathways mediates the function of CIP in cardiac protection. (A) A volcano plot of all detected genes in RNA-seq. Each dot represent one gene and the darker grey dots indicate dys-regulated genes in TAC-CIP-OE hearts comparing to TAC-Ctrl hearts. (B) A Hierarchical clustering heatmap of 1236 dys-regulated genes in all groups. (C) Gene ontology analysis of 444 up-regulated (p<0.05) and 792 down-regulated (p<0.05) genes in TAC-CIP-OE hearts. The GO terms are ranked by the adjusted P values. (D) Ingenuity Pathway Analysis (IPA) of upstream regulators of dys-regulated genes in CIP-OE heart after 8 weeks of TAC operation. (E) qRT-PCR validation of dys-regulated genes downstream of IGF1R, Rictor or TGF1B. N=3 for each group. The significance between groups was tested with 1-way ANOVA with post-hoc Tukey's test. *P<0.05; **P<0.01.

FIGS. 11A-O. Skeletal muscle CIP is essential for myonuclear positioning in vitro. (A) schematics for mouse CIP genomic locus and cardiac and skeletal muscle predominant mRNA isoforms. Gray box indicates non-translated sequence. Open box indicates ORF. (B) CIP expression levels across a broad range of human tissues. The graph shows highest CIP expression in striated muscles. Data are derived from GTEx Consortium. (C) RNA-seq signal as density of mapped reads corresponding to muscle specific exons derived from ENCODE project. (D) Heatmap of skeletal muscle-specific CIP and common CIP expression levels in various tissues. Data are derived from GTEx Consortium. (E) Control and CIP siRNA infected C2C12 myoblasts differentiated for 5 days and stained with MF20 antibody (myosin) and Lamin A/C. Arrows indicate myonuclear misalignment in myotubes. Scale bar, 50 μm. (F) Quantification of myonuclei clustering in control and CIP siRNA treated C2C12 myoblasts. *, p<0.05; **, p<0.001. (G) Diagram of CRISPR/Cas9 mediated knockout of CIP gene. (H) PCR-based genotyping to verify correct targeting of the CIP gene. Shown are SEQ ID NOs:69-70. (I) Western blot showing successful knockout of CIP protein. (J) Control and CIP knockout C2C12 myoblasts differentiated for 5 days and stained with MF20 antibody (myosin) and Lamin A/C show that CIP is required for myonuclear alignment in myotubes. Scale bar, 50 μm. (K) Quantification of myonuclear clustering in control and CRISPR/Cas9-CIP-KO C2C12 myoblast cultures. (n=3 pairs). *, p<0.01. (L and M), Phase contrast and immunofluorescence images of WT and CIP KO primary myoblasts differentiated for 2 days and stained with MF20 antibody (myosin) and Lamin A/C, showing extensive myonuclei clustering in primary CIP-KO myofibers. Scale bar, 50 μm. (N) Quantification of myonuclei clustering in WT and CIP-KO primary myoblast cultures. (n=3 pairs). *, p<0.001. (0), Infection of primary CIP KO myotubes with SK-CIP_EGFP adenovirus rescues the nuclei clustering and results in linear alignment of myonuclei.

FIGS. 12A-L. Skeletal muscle CIP is essential for myonuclear positioning in vivo. (A) Hematoxylin and eosin (H&E) staining of cross sections of 3-month-old WT and CIP-KO TA muscles. (B) Hematoxylin and eosin (H&E) staining of longitudinal sections of 8-month-old WT and CIP-KO TA muscles show peripheral myonuclei clustering in CIP-KO myofibers. Insert is magnified view of white box region. Scale bar, 50 μm. (C) Binary images of nuclear staining of CIP WT and KO TA muscle. Scale bar, 50 μm. (D) Quantification of Fractal Dimension analysis of WT and KO TA muscle nuclear staining, showing higher disorganization in KO muscles. (E) Isolated WT and CIP-KO EDL muscle fibers stained with MF20 (Myosin) and DAPI show aberrant myonuclei clustering. Scale Bar, 50 (F) Quantification of myonuclei clustering in WT and CIP-KO isolated EDL muscle fibers shows that most of the CIP-KO muscle fibers contain more than three myonuclei clusters. n represents the number of muscle fibers. (G) Isolated WT and CIP-KO EDL muscle fibers stained with MF20 (Myosin), Alexa-488 α-bungarotoxin, and DAPI show aberrant myonuclei anchoring at the neuromuscular junction (NMJ). Scale Bar, 50 μm. (H) Quantification of aberrant myonuclei anchoring at NMJs shows a reduction in the number of myonuclei that are well surrounded by AChR in CIP-KO myofibers. (I) Isolated WT and CIP-KO EDL muscle fibers stained with DAPI show aberrant myonuclei clustering at the myotendinous junction (MTJ). Scale Bar, 50 μm. (J) Quantification of myonuclei clustering at MTJs in WT and CIP-KO isolated EDL muscle fibers shows significantly more aberrant myonuclei positioning in CIP-KO muscle fibers. (K) Cardiotoxin-injured and Hematoxylin and eosin (H&E) stained 3-month-old TA muscles after three weeks of regeneration shows the aggregation of centrally located myonuclei in CIP-KO mice. Scale Bar, 50 (L) Quantification of aggregated centrally located nuclei in regenerated TA muscle after cardiotoxin injury shows a drastic increase in the percentage of myofibers with aggregated centrally located nuclei in CIP-KO muscle. N=4 animals for each genotype.

FIGS. 13A-I. Skeletal muscle CIP is required for intracellular muscle nuclei organization in muscle diseases, and loss of CIP causes a more severe muscular dystrophy in mdx mice. (A) Photographs of 1 year old mdx and CIP/mdx double knockout mice to illustrate the smaller body size and progressive weakness of DKO mice. (B) micro-CT of 1-year old WT, CIP-KO, mdx and CIP/MDX DKO mice showing severe kyphosis in DKO mice. Arrow indicates the inability of DKO mice to fully extend hind limbs due to muscle weakness. (C) Hematoxylin and Eosin (H&E) and Sirius Red/Fast Green staining of diaphragm cross sections from 1-year old mdx and CIP/mdx dKO mice. Scale bar, 50 pin. (D) Quantification of Evans blue dye-positive fibers in TA muscles of mdx and CIP/mdx dKO mice (n=6 for each group). Whole TA muscle stitched images are shown here for representational purposes. (E) Confocal images of TA muscle stained for laminin to mark myofibers, DAPI to mark nuclei, from mdx and CIP/mdx DKO mice. Both cross (left panels) and longitudinal sectional images (right panels) reveal a significant higher degree of disorganized centrally located myonuclei positioning in DKO mice (arrows). Scale bar, 100 μm. (F) Quantification of percentage of myofibers with 3 clustered centrally located nuclei in mdx and CIP/mdx mice respectively. Animal numbers for each group are indicated on each bar. (G) Representative Actitrack activity plots of the 4 mouse genotypes measured before (Pre-exercise) and after (Post-exercise) forced downhill treadmill running. The thin line represents the mouse's distance traveled, while the dark lines represent instances of rearing (vertical activity). (H) Summary graph plotting the total distance and vertical activity of the 4 genotypes before and after downhill running respectively. Animal number for each group is indicated under each bar. (I) Limb grip strength measurement of mice from the 4 genotypes, measured in grams. Animal numbers for each group are indicated on each bar.

FIGS. 14A-C. Treatment of CIP-KO/Mdx mice with Nox4 inhibitor (GKT137831). (A) Schematic illustration of protocol for experiments. (B, C) Echocardiography measurement of cardiac function of CIP-KO/mdx dko mice in the young group (B, Starting at 3-months old) and Old group (C, Starting at 5-months old) before (Pre-) or after treated with control (C-) or Nox4 inhibitor (I-) at different time points (1, 2, 3, 4 and 5 months). EF, ejection fraction.

FIGS. 15A-E. Treatment of Utrn/Mdx mice with Nox4 inhibitor (GKT137831). (A) Schematic illustration of protocol for experiments. (B, C) Echocardiography measurement of cardiac function of Utm/Mdx dKO mice in the young group (B, Starting at 3-months old) and Old group (C, Starting at 5-months old) before (Pre-) or after treated with control (C-) or Nox4 inhibitor (I-) at different time points (2, 4 and 8 weeks). EF, ejection fraction. (D) Fast green/Sirius red staining of heart section of Utrn/mdx dko mice 8 weeks after being treated with control or Nox4 inhibitor. Quantification of fibrosis is present at right panel. (E) Quantitative real time PCR detecting the expression of fibronectin in the hearts of Utrn/mdx dko mice 8 weeks after being treated with control or Nox4 inhibitor.

### DETAILED DESCRIPTION

We have previously identified CIP as a cardiac and skeletal muscle-specifically expressed protein¹¹, an alternatively spliced isoform of a recently reported protein called MLIP^{12,13}. We showed that CIP plays a key role in cardiac remodeling in response to pathophysiological stress¹⁴. Clinical investigation further revealed that mutation of the CIP gene was associated with dilated cardiomyopathy⁵⁰. Although overexpression of CIP inhibited hypertrophic growth of cardiomyocytes¹¹, whether CIP is able to inhibit or even reverse cardiac remodeling after cardiac disease has been established remains unanswered. In this study, we show how CIP expression is regulated in cardiomyocytes and diseased human hearts. Furthermore, we demonstrate that CIP inhibits cardiac remodeling and protects the heart from HF after cardiac hypertrophy has been established, providing a proof-of-concept for the clinical potential of CIP in the treatment of cardiac hypertrophy and HF. In addition, we report an important function of CIP in dystrophic cardiomyopathy. Genetic studies show that loss of CIP accelerates the progress of cardiomyopathy and heart failure in mouse models of DMD. Most importantly, overexpression of CIP protects the heart from the development of pathological dystrophic cardiomyopathy in mdx mice. In addition, we show that CIP is a novel regulator of nuclear positioning in mammalian skeletal muscle and plays an important role in skeletal muscle and muscular dystrophy.

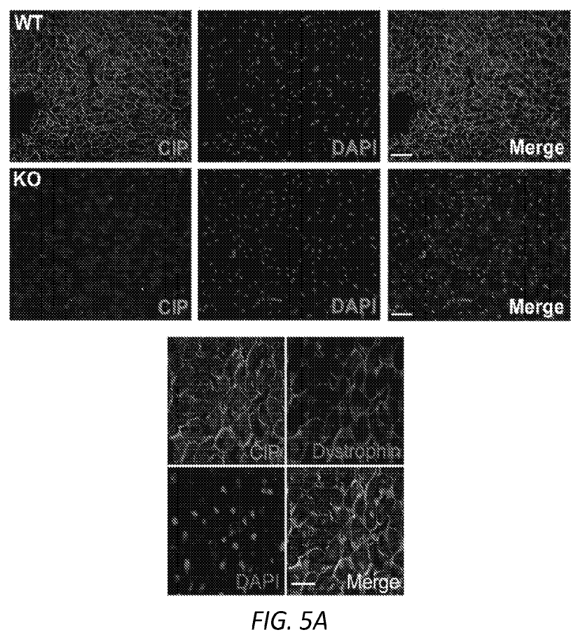

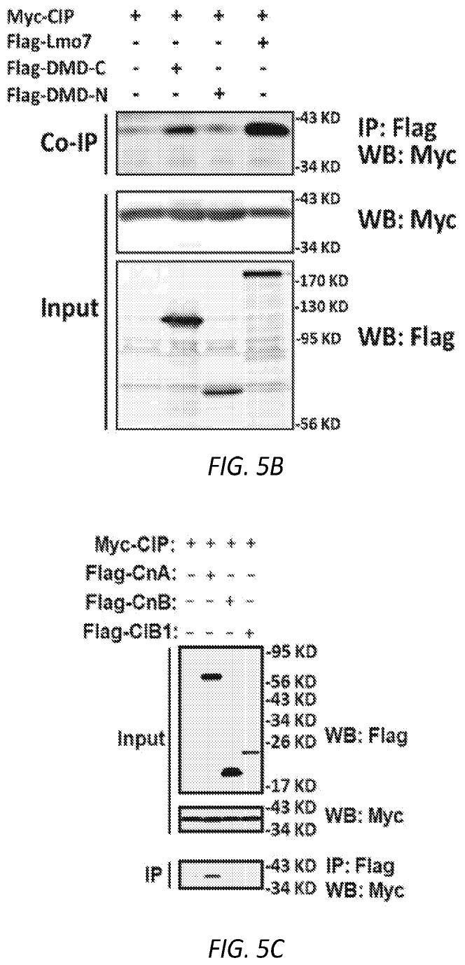

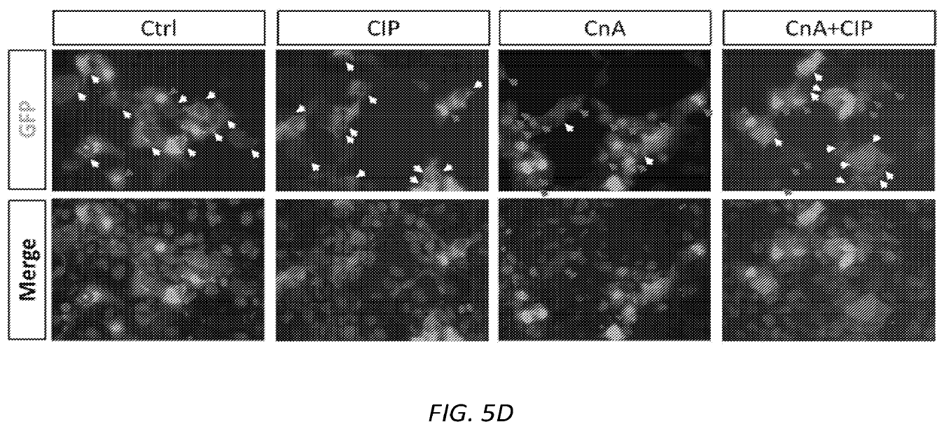

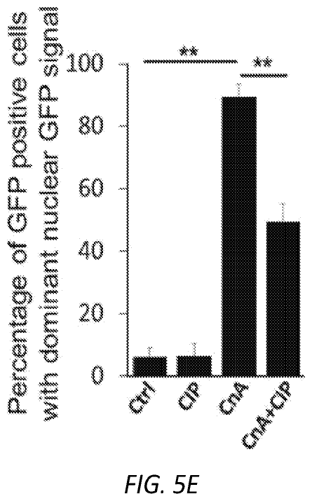

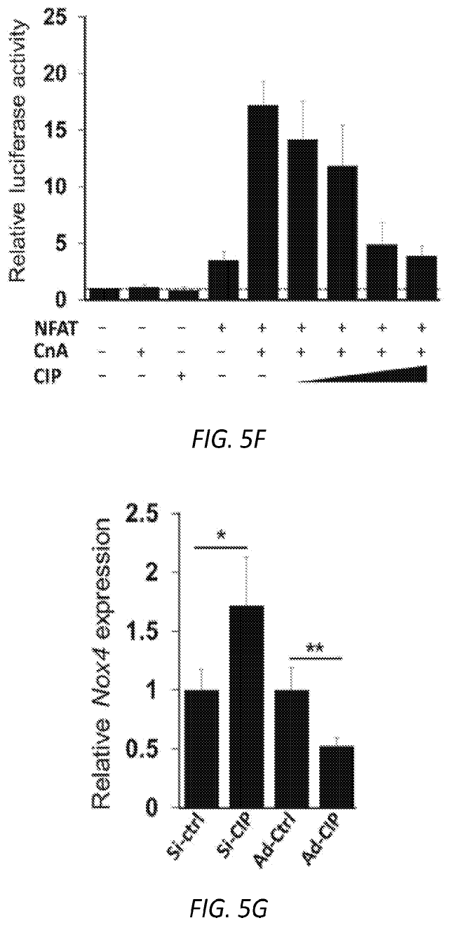

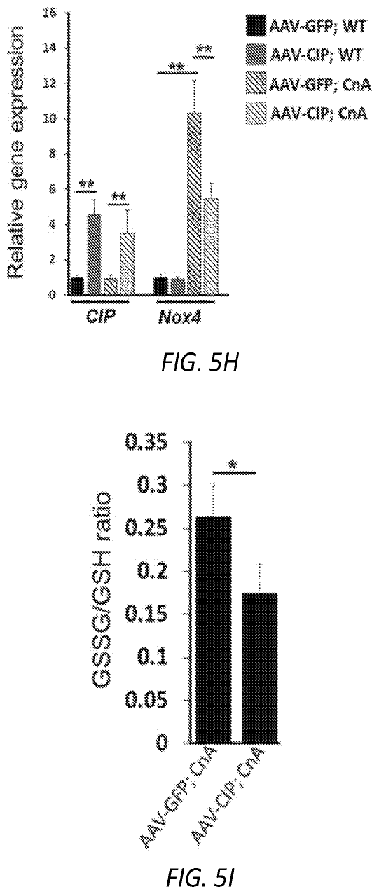

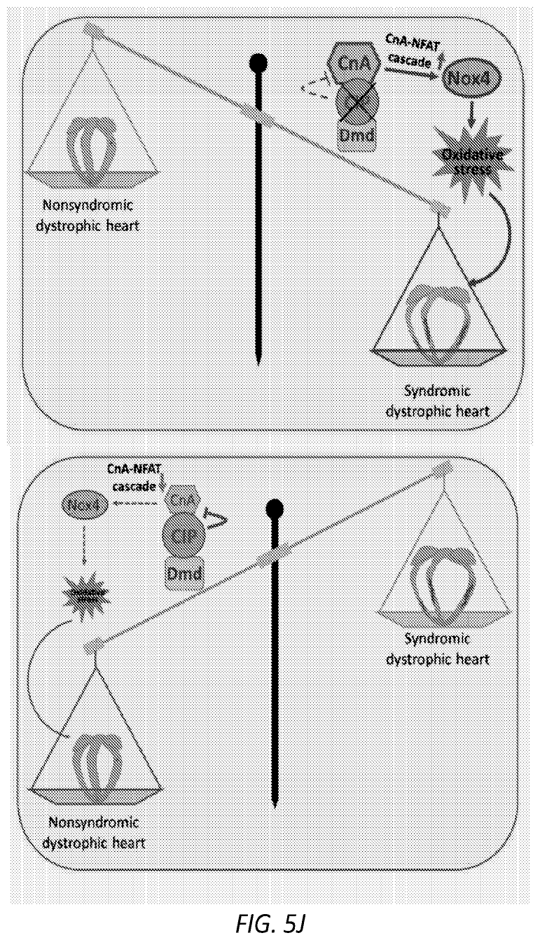

Loss of dystrophin, an important structure protein on the cell membrane of cardiac and skeletal muscles, leading to an increase in the cell permeability, fibrosis and inflammatory response of myocytes, eventually results in loss of myocytes and a decrease of muscle contractility^{9, 10, 35-37}. This creates substantial challenge for the potential treatment of muscular dystrophy^{3, 38, 39}. Our study demonstrates that cardiac-expressed CIP ameliorates cardiac function in dystrophic heart, by modulating the expression and function of Nox4 and oxidative stress by regulating the calcineurin-NFAT pathway (FIG. 5J). We further reveal that increased oxidative stress is a causative factor of dystrophic cardiomyopathy. This study identifies CIP as a novel regulator of dystrophic cardiomyopathy and suggests it as a novel therapeutic target to treat this disease⁴⁰.

The pathophysiology and cell biology of muscular dystrophy, including that of cardiomyopathy and heart failure, has been extensively investigated and understood. However, the underlying molecular signature and mechanisms remain to be fully understood. Using unique animal models we have created that exhibit many features of dystrophic cardiomyopathy, our gene expression signature analyses revealed key molecular pathways that are closely associated with the conditions of dystrophic cardiomyopathy. Strikingly, genes related to extracellular matrix are among the most significantly changed in dystrophic hearts, consistent with the view that fibrosis is a “default” end point of dystrophic cardiomyopathy^{9, 23, 39, 41}. Similarly, we found that genes related to calcium signature are tightly associated with the pathological condition of dystrophic hearts⁴². These results enable us to better understand the molecular nature of this disease and provides useful bio-markers for the early diagnosis of dystrophic cardiomyopathy.

Numerous prior studies have linked the oxidative stress pathway to cardiomyopathy. Increased expression of Nox4, which is a major resource of oxidative response²⁴, and other oxidative pathway genes was found in Duchenne cardiomyopathy⁴³⁻⁴⁶. Altered Nox2 and Nox4 expression was also associated with other types of cardiomyopathy^{47, 48}. During cardiac hypertrophy, increased Nox4 expression is associated with apoptosis and defects in mitochondria⁴⁹. However, it remains unclear about how Nox4 expression is regulated in dystrophic cardiomyopathy. Our study suggests that mutation in both dystrophin and CIP triggered the calcineurin pathway, activating NFACT, resulting in an increase expression and function of Nox4. Our study also provided direct evidence to demonstrate ectopic overexpression of Nox4 in the heart activates the oxidative stress pathway and leads to cardiomyopathy.

Previous studies have suggested that in Duchenne muscular dystrophy patients, the pathophysiological mechanisms involved in cardiac myocytes seem to differ significantly from those in skeletal myofibers^{9, 10}. Intriguingly, we also observed that a skeletal muscle-specific CIP isoform, skCIP, regulates myonuclear positioning in skeletal myoblasts and myotubes, thereby modulating skeletal muscle function and regeneration. Interestingly, we have found that loss of CIP in adult cardiomyocytes also affects the positioning of bi-nuclei, suggesting that CIP may also plays a role in myonuclei positioning in cardiomyocytes. Together, these studies indicate that CIP is a novel regulator of myocyte function, cardiomyopathy and muscular dystrophy. Therefore, our findings indicate that cardiac- and skeletal muscle-specific CIP isoforms participate in the regulation of myocyte function in the heart and skeletal muscle through different mechanisms. Given that a recent study found that human CIP was mutated in patients with dilated cardiomyopathy and heart failure⁵⁰, CIP could be a novel target for therapeutic treatment of dystrophic cardiomyopathy.

As shown herein, deletion of CIP in mdx mouse, a mouse model of DMD disease, caused severe cardiac defects and resulted in severe muscular dystrophy. Conversely, transgenic overexpression of CIP in the heart and skeletal muscle protects the heart from the development of heart failure, demonstrating a beneficial role of CIP in DMD. We have also identified a skeletal much isoform of CIP (skCIP) and found that mutation of CIP leads to defects in myonuclear positioning in skeletal muscle. We identified Nox4 as an important downstream mediator of the CIP function in the heart. When applied Nox4 inhibitor GKT137831 to mdx/CIP-KO mice, we observed that this inhibitor potently prevents the development of heart failure. Together, these studies indicate: 1) CIP is an important regulator of dystrophic cardiomyopathy; 2) CIP is also important for skeletal muscle function; 3) Inhibition of Nox4 using chemical inhibitors could protect the heart from the development of heart failure in dystrophy patients.

Thus, provided herein are methods for treating or reducing risk of developing cardiomyopathy/heart failure. The methods include administering a therapeutically effective amount of a Nox4 inhibitor. The methods can be used in mammalian subjects, e.g., human or non-human veterinary subjects. In some embodiments, the subjects have muscular dystrophy, e.g., DMD. The methods can include treating subjects who are at risk of, but who do not yet have (e.g., have not yet been diagnosed with or have no symptoms or clinical signs of, e.g., have no or minimal left ventricular dysfunction), dystrophic cardiomyopathy. The methods can also be used to delay or reduce the risk of progression from cardiac hypertrophy to heart failure, i.e., in subjects who already have cardiac hypertrophy.

Also provided herein are methods for treating or reducing risk of developing skeletal muscle dysfunction, e.g., dystrophy. The methods include administering a therapeutically effective amount of a Nox4 inhibitor. The methods can be used in mammalian subjects, e.g., human or non-human veterinary subjects. In some embodiments, the subjects have muscular dystrophy, e.g., DMD. In some embodiments, the methods reduce weakness or improve strength, or reduce the rate or risk of developing muscle weakness.

In some embodiments, the methods can include using imaging methods such as echocardiography or cardiovascular magnetic resonance (CMR) to identify a subject who has the earliest signs of cardiac involvement, including left ventricular (LV) strain defects and myocardial fibrosis, which appear before the onset of LV systolic dysfunction (see, e.g., JAMA Cardiol. 2017; 2(2):199. doi:10.1001/jamacardio.2016.4910); Silva et al., JAMA Cardiol. doi:10.1001/jamacardio.2016.4801). In some embodiments, the subject has sinus tachycardia, atrial arrhythmias, including atrial fibrillation, atrial flutter, and atrial tachycardias, with left ventricular ejection fraction (LVEF)<35%. In some embodiments, the subject has cardiac hypertrophy. In some embodiments, the methods include identifying and/or selecting subjects for treatment with a method described herein based on the presence of cardiac involvement or muscular dystrophy.

### Nox4 Inhibitors

The methods described herein include administration of one or more Nox4 inhibitors.

A number of Nox4 inhibitors are known in the art, including GKT137831 (Setanaxib, Genkyotex); GKT136901; GSK2795039; VAS2870; perhexiline; VAS3947; compound 87 (2-(2-chlorophenyl)-5-[(1-methylpyrazol-3-yl)methyl]-4-[[methyl(pyridin-3-ylmethyl)amino]methyl]-1H-pyrazolo[4,3-c]pyridine-3,6-dione; PMID 20942471); compound 7c (10-benzyl-2-(2-chlorophenyl)-7,8,9,11-tetrahydro-3H-pyrazolo[4,5]pyrido[5,6-a][1,4]diazepine-1,5-dione; Gaggini et al., Bioorg Med Chem. 2011 Dec. 1; 19(23):6989-99); APX-115; VAS2870; fulvene-5; grindelic acid; phenantridinones (Borbély et al., J Med Chem. 2010 Sep. 23; 53(18):6758-62); fluvenazine; DPI; suramin; ebselen; perhexiline; perhenazine; fluphenazine; and tertiary sulfonylureas (23-25) (Xu et al., Bioorg Med Chem. 2018 Mar. 1; 26(5):989-998). See also Reis et al., Redox Biol. 2020 May; 32: 101466; Xu et al., Bioorg Med Chem. 2018 Mar. 1; 26(5):989-998.

### Pharmaceutical Compositions and Methods of Administration

The methods described herein include the use of pharmaceutical compositions comprising or consisting of a Nox4 inhibitor as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, in addition to the Nox4 inhibitor, in some embodiments, no other active ingredients are present or used in the compositions or methods described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

### Example 1. Cardiac CIP Protein Ameliorates Dystrophic Cardiomyopathy Methods

The following materials and methods were used in the following example.

Mice. All experimental procedures involving animals in this study were reviewed and approved by the Institutional Animal Care and Use Committee at the Boston Children's Hospital. CIP-KO and CIP-KI-flox mice were described previously¹⁴ and bred with mdx mice¹⁵. To obtain CIP-OE mice, the CIP-KI-flox mice, which have a Rosa-CIP allele (containing a loxed stop codon) were bled with MCK-Cre mice¹⁶ to excise the stop codon and activate the CIP transgene in the heart and skeletal muscle. Calcineurin transgenic mice (CnA-tg)¹⁷ were used in this study. Compound mutant mice on mixed genetic backgrounds were used and all comparisons used littermates as controls.
Measurement of cardiac function by echocardiography. Echocardiographic measurements were performed on mice using a Visual Sonics Vevo® 2100 Imaging System (Visual Sonics, Toronto, Canada) with a 40 MHz MicroScan transducer (model MS-550D). Mice were anesthetized with isoflurane (2.5% isoflurane for induction and 0.1% for maintenance). Heart rate and left ventricular (LV) dimensions, including diastolic and systolic wall thicknesses, LV end-diastolic and end-systolic chamber dimensions were measured from 2-D short-axis under M-mode tracings at the level of the papillary muscle. LV mass and functional parameters such as percentage of fractional shortening (FS %) and ejection fraction (EF %) were calculated using the above primary measurements and accompanying software.
Generation and administration of adeno-associated virus (serotype 9). GPF-tagged mouse CIP cDNA, Flag-tagged mouse Nox4 cDNA and GFP were separately cloned into ITR-containing AAV plasmid (Penn Vector Core P1967) harboring the chicken cardiac TNT promoter, to yield constructs, pAAV9-cTnT-CIP, pAAV9-cTnT-Nox4 and pAAV9-cTnT-GFP, respectively. AAV was packaged using AAV9:Rep-Cap and pAd:deltaF6 (Penn Vector Core) as described¹⁸. AAV9 was packaged in 293T cells with AAV9:Rep-Cap and pAd deltaF6, then purified and concentrated by gradient centrifugation. AAV9 titer was determined by quantitative PCR. AAV9 virus (4×10¹¹ virus genome/animal) were injected into postnatal day 2 CnA-tg pups, mdx pups or their control littermates with subcutaneous injection. Hearts were harvest at the age of 4 weeks for CnA-tg mice and at the age of 2 months for mdx mice.
Haematoxylin and eosin staining and fast green/Sirius red collagen staining. Mouse heart tissues were dissected out, rinsed with PBS and fixed in 4% paraformaldehyde (pH 8.0) overnight. After dehydration through a series of ethanol baths, samples were embedded in paraffin wax according to standard laboratory procedures. Sections of 5 μm were stained with haematoxylin and eosin (H&E) for routine histological examination with light microscope. For Sirius red/fast green collagen staining sections were fixed with pre-warmed Bouins' solution, 55° C. for 1 hour then washed in running water. Sections were stained in 0.1% fast green solution for 10 minutes then washed with 1% acetic acid for 2 minutes. After rinsing in tape water, sections were stained in 0.1% Sirius resolution for 30 minutes. After staining, sections were dehydrated and cleared with xylene. The images were examined with light scope and quantified with ImageJ software.
Immunofluorescence. Mouse heart tissues were dissected out, collected and fixed in 4% PFA at 4° C. for 4 hours. After washing in PBS, samples were treated in 15% and 30% sucrose for 2 hours each and embedded in OCT. About 5-8 μm cryostat sections were collected on positively charged slides. Sections were washed in PBS, blocked in 5% serum/PBS, and subjected to immunostaining. Antibody sources were as follows: Anti-CIP (1:200, 21st Century Biochemical, customized); Anti-Dystrophin (1: 100, Developmental Studies Hybridoma Bank, MANDRA1-7A10) Anti-Flag (1:200, Sigma-Aldrich, F1804). Alexa-488 and 594 secondary antibodies (Life technologies). Fluorescently stained cells were counterstained with DAPI and imaged with an FV1000 confocal microscope (Olympus).
Isolation of cardiomyocytes from adult mice. Adult mouse cardiomyocytes were isolated using a previously described procedure¹⁹ with minor modifications. Briefly, following perfusion and digestion of the heart with collagenase II (Worthington Biochemical Corp, Lakewood, N.J.), dissociated cells (myocytes and non-myocytes) were sedimented by gravity. The bottom layer is rich in adult cardiomyocytes. Cardiomyocytes were then collected and fixed with 4% PFA for immunofluorescence.
Cardiomyocyte culture. Neonatal mouse and rat cardiomyocytes were prepared as previously described²⁰. Briefly, neonatal cardiomyocytes were isolated by enzymatic disassociation of 1-day old neonatal mouse or rat heart with the Neonatal Cardiomyocyte Isolation System (Cellutron Life Technology). Cardiomyocytes were plated for 2 hours to remove fibroblasts. Cells were then plated on 1% gelatin-coated plates in medium containing 10% horse serum and 5% fetal calf serum (FCS). Eighteen hours after plating, cells were changed into serum-free medium and infected with adenovirus (25 MOI) for 24 hours. For the treatment of siRNA, fifty (50) nM of siRNA targeting CIP transcript (Si-CIP) and control siRNA (from Dharmacon) were transfected into cardiomyocyte by using Lipofectamine RNAiMAX transfection reagent. Six hours later, medium with transfection reagent were removed. Cells were then treated with phenylephrine (PE, 20 μM) or Angiotensin II (ANG II, 1 μM). Cells were harvested 24 hours after PE treatment for RNA isolation or 4 hours after ANG II treatment for measurement of GSH/GSSG ratio.
Quantitative RT-PCR and Western blot analysis. Total RNAs were isolated using Trizol Reagent (Life technologies) from cells and tissue samples. For quantitative RT-PCR, 2.0 μg RNA samples were reverse-transcribed to cDNA by using random hexamers and MMLV reverse transcriptase (Life technologies) in 20 μl reaction. In each analysis, 0.1 μl cDNA pool was used for quantitative PCR. The relative expression of interested genes is normalized to the expression of 18S rRNA or β-actin. For Western blot analyses, protein samples were cleared by 10,000×g centrifugation for 10 min. Samples were subsequently analyzed by SDS/PAGE and transferred to PVDF membranes that were incubated with Odyssey Blocking Buffer (LI-COR) and Anti-CIP (1:2,000, 21st Century Biochemical, customized); Anti-β-tubulin (1:10,000, Sigma-Aldrich, T0198); Anti-Flag (1:5,000, Sigma-Aldrich, F7425); Anti-Myc (1:5,000, Sigma-Aldrich, C3956); Anti-Lmo7 (1:1,000, Santa Cruz Biotechnology, sc-376807); Anti-HSPA9 (1:1,000, Santa Cruz Biotechnology, sc-133137); Anti-α-actinin (1:1,000, Sigma-Aldrich, A7811); Anti-NFAT4 (1:500, Santa Cruz Biotechnology, sc-8405); Anti-phospho-NFAT4 (1:500, Abcam, ab59204) or Anti-dystrophin (1:500, Developmental Studies Hybridoma Bank, MANDRA1-7A10) overnight at 4° C. and then washed three times with PBS buffer before adding IgG secondary antibody. Specific protein bands were visualized by Odyssey CLx imager (LI-COR).
Coimmunoprecipitation assays. HEK293 cells were transiently transfected with plasmids using Lipofectamine 3000 (Invitrogen). Cells were harvested 48 hours after transfection in lysis buffer composed of PBS containing 0.5% Triton X-100, 1 mM EDTA, 1 mM PMSF, and complete protease inhibitors (Roche). For heart tissue, neonatal CIP-KO and control hearts were lysed with above-mentioned lysis buffer. After a brief sonication and removal of debris by centrifugation, proteins were precipitated with Anti-Flag, Anti-Myc or Anti-CIP antibodies and protein A/G beads and analyzed by Western blotting with indicated antibodies.
In vitro GST protein-binding assays. Plasmids encoding a GST fusion with CIP were transformed into BL21 plus cells (Stratagene). The cells were grown at 37° C. in 2×YT medium to an optical density of 1.0. Isopropyl-β-D-thiogalactopyranoside (50 μM) was then added to the culture to induce protein expression. After being shaken at room temperature for 4 h, the cells were harvested and the GST protein was purified with glutathione beads. Glutathione beads conjugated with GST fusion protein were incubated with wild-type heart lysate at 4° C. for 6 hours in 500 μl of GST-binding buffer (20 mM Tris, pH 7.3/150 mM NaCl/0.5% Nonidet P-40/protease inhibitor/1 mM phenylmethylsulfonyl fluoride). The beads were washed three times with GST binding buffer. 25 μl of SDS loading buffer was then added to the beads. After boiling, 25 μl was loaded onto an SDS/PAGE gel to separate the CIP-binding proteins. Gel was then stained with Coomassie blue. Stained protein bands were cut out and followed by Mass spectrometry analysis.
Constructs, cell culture, and luciferase reporter assays. COS7 and HEK293T cells were cultured in DMEM supplemented with 10% FBS in a 5% CO2 atmosphere at 37° C. Firefly luciferase reporter constructs fused with the 3×NFAT binding sequence in the upstream region was purchased from Addgene. Transfections were performed with Lipofectamine 3000 (Invitrogen) reagents according to manufacturer's instruction. Cells were co-transfected with NFAT luciferase reporter, Renilla luciferase reporter (normalizing control) and other indicated plasmids. 48 hours after transfection, cell extracts were prepared and luciferase activity was determined. For luciferase assay, normalized luciferase activity from triplicate samples in 12-well plates relative to Renilla luciferase activity was calculated, and the results are expressed as fold activation over the value relative to the control (NFAT luciferase reporter and empty pcDNA). To determine the subcellular location of GFP-NFAT fusion protein, COS7 cells were co-transfected with GFP-tagged NFAT plasmid (Addgene) and other indicated plasmids. Cells were cultured for 24 hours before taking the live cell images.
GSH/GSSG measurement. Measurement of GSH/GSSG was performed with GSH/GSSG Ratio Detection Assay Kit (ab138881) according to manufacturer's instruction.
RNA-seq data analysis. Raw reads were mapped to UCSC mm9 using tophat 2.0 [1]. RNA fragment was counted by htseq-count [2]. RNA fragment was further normalized per kilobase of exon per million mapped fragment (FPKM). Differentially expressed gene was calculated using DE-seq [3], and fold change>0.5 and p-value<0.05 were used as parameter.
Heatmap of gene expression level. Log 2 fold change was calculated by fragments in treated groups over control groups. Positive value stands for higher expression level in treated groups and vice versa. Heatmap was clustered using hierarchy cluster method, and Euclidean distance, complete linkages were used as the parameter.
Principal component analysis. All expressed genes in the genome from RNA-seq analyses were used as the signature of each group. Principal component analysis (PCA) method was used to find top principal components among groups. We found that top 2 principal components were sufficient to reach a cumulative energy of 0.98. Principal component 1 and 2 were plotted as x-axis and y-axis in 2-D coordinates.
Statistics. Values are reported as means±STD unless indicated otherwise. An analysis of variance (ANOVA) analysis followed by Dunnett's Post-Hoc testing was used to evaluate the statistical significance for multiple-group comparisons. In addition, the 2-tailed Mann-Whitney U test was used for 2-group comparisons. Values of P<0.05 were considered statistically significant.

### Results

### Loss of CIP Accelerates the Development of Dystrophic Cardiomyopathy

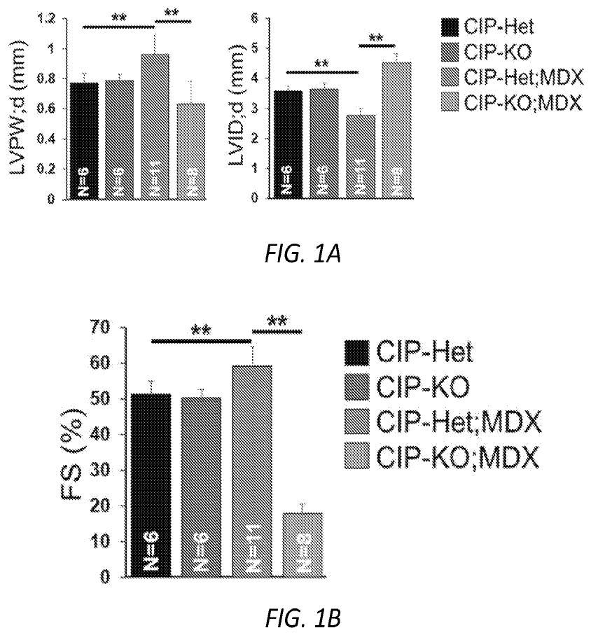

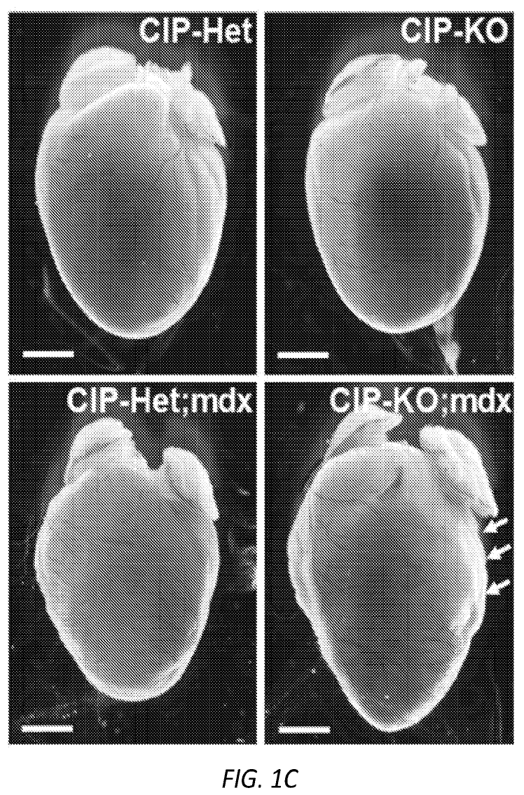

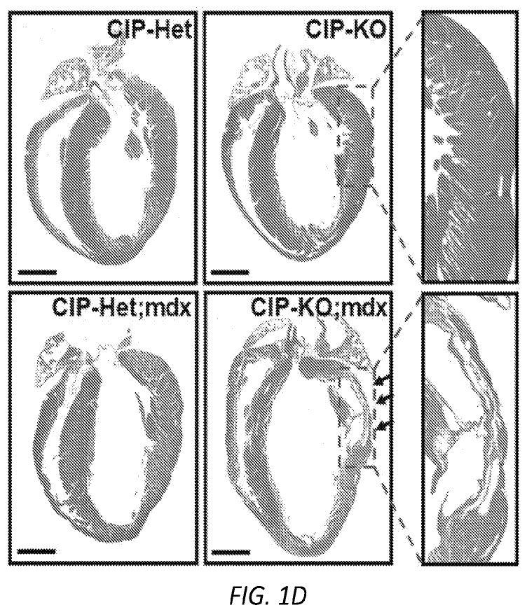

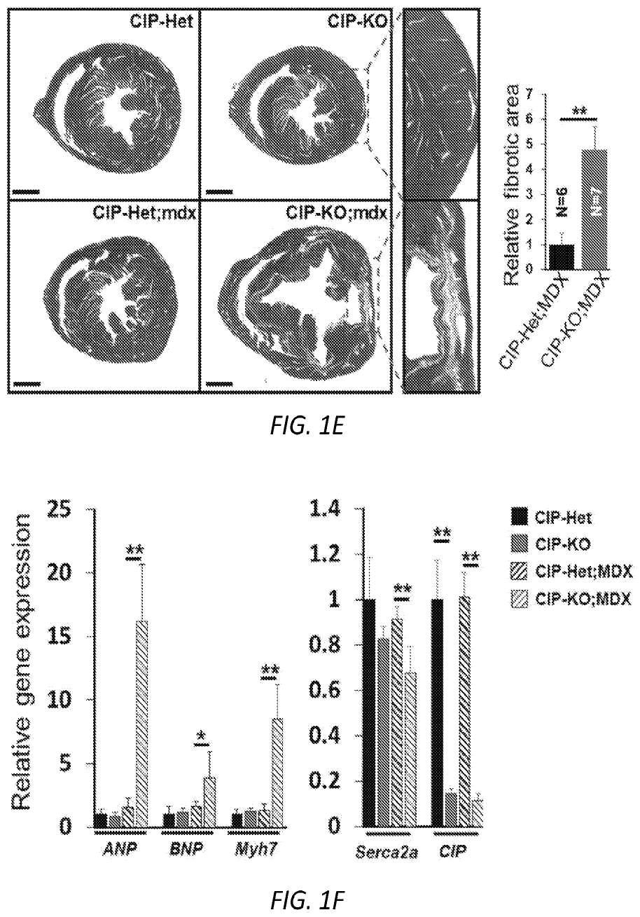

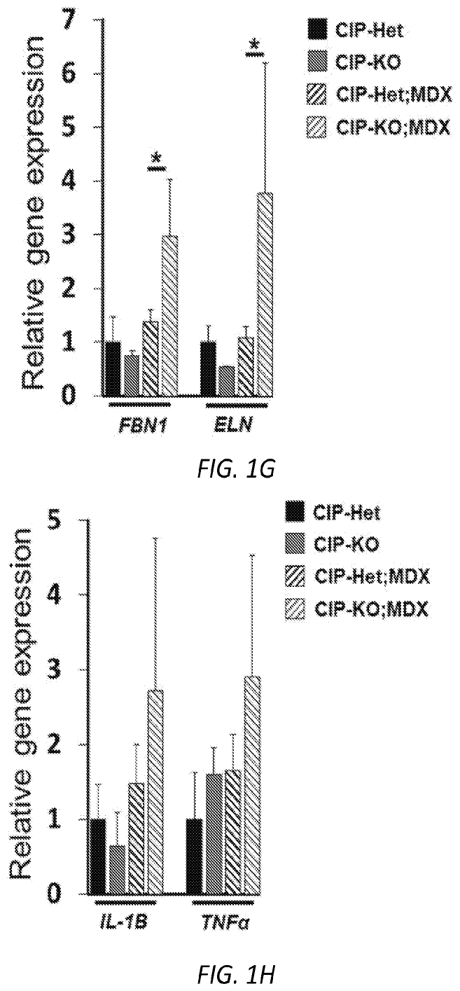

To test the hypothesis that genetic interaction between CIP and dystrophin modulates muscular dystrophy, we crossed the CIP knockout (CIP-KO) mouse with the mdx mouse, which harbors a dystrophin mutation and represents a mouse model of human DMD, to generate a CIP-KO; mdx double knockout (dKO) mouse. CIP-KO mice appear normal without detectable cardiac defects up to 12 months of age¹⁴, and mdx mice only start to display mild cardiomyopathy at 10 months of age^{21, 22.} Strikingly, we found that the dKO mice exhibit severe cardiomyopathy as early as 3 months of age. Echocardiographic measurements showed that left ventricular posterior wall is much thinner and left ventricular internal diameter is significantly enlarged in dKO mice, when compared with control groups (FIG. 1A). Fractional shortening in dKO mice decreased to below 20% (FIG. 1B), indicating that they suffer from severe dilated cardiomyopathy and heart failure, which is commonly observed in DMD patients^{9,10}. Morphological and histological examination verified that dKO hearts were significantly enlarged and dilated (FIGS. 1C, 1D). Intriguingly, focal myocardial lesions, often observed in DMD hearts²³, are often found in the left ventricle of dKO hearts (Arrows in FIGS. 1C, 1D). Sirius Red/Fast Green staining showed a dramatic increase in cardiac fibrosis in dKO hearts (FIG. 1E).

We examined molecular markers for cardiomyopathy and heart failure, and found that the expression levels of ANF, BNP and Myh7 were all elevated in the hearts of dKO mice (FIG. 1F). Notably, the expression of these cardiomyopathy marker genes was not altered in the hearts of control mice (CIP-Het, CIP-KO or CIP-Het; mdx), further supporting the view that CIP is required for normal cardiac function in dystrophic mice. Consistent with increased fibrosis, we detected increased expression of fibrosis markers FBN1 and ELN in dKO hearts (FIG. 1G). Cardiac fibrosis is often a result of cardiomyocyte death. Indeed, the expression of cell death-related genes IL-1 and TNF were also elevated in dKO hearts (FIG. 1H). Collectively, these data indicate that loss-of-function of CIP accelerates the disease progression of dystrophic cardiomyopathy.

Transgenic Overexpression of CIP Protects the Heart from the Development of Dystrophic Cardiomyopathy

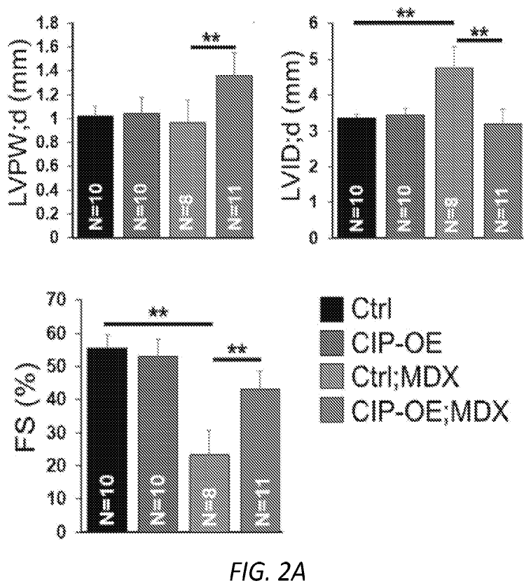

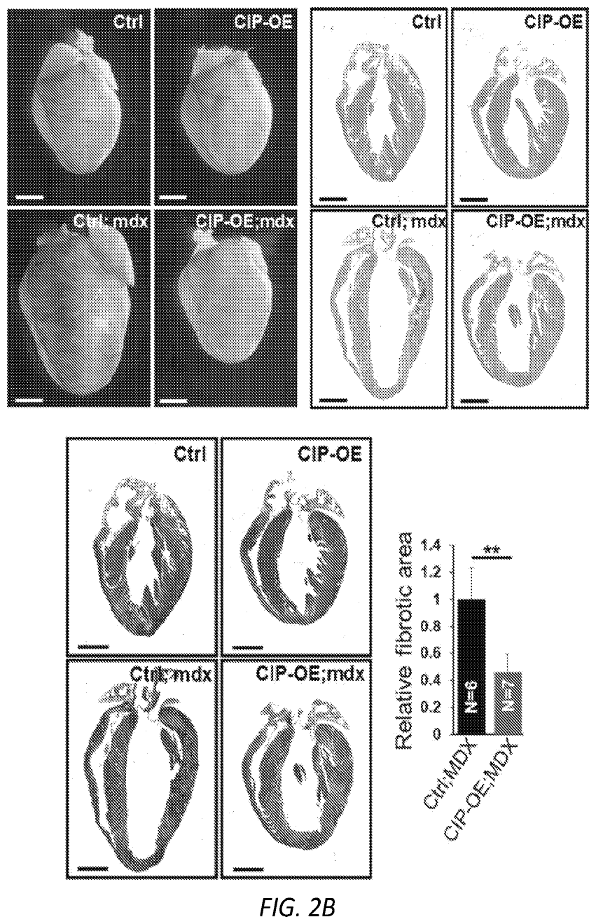

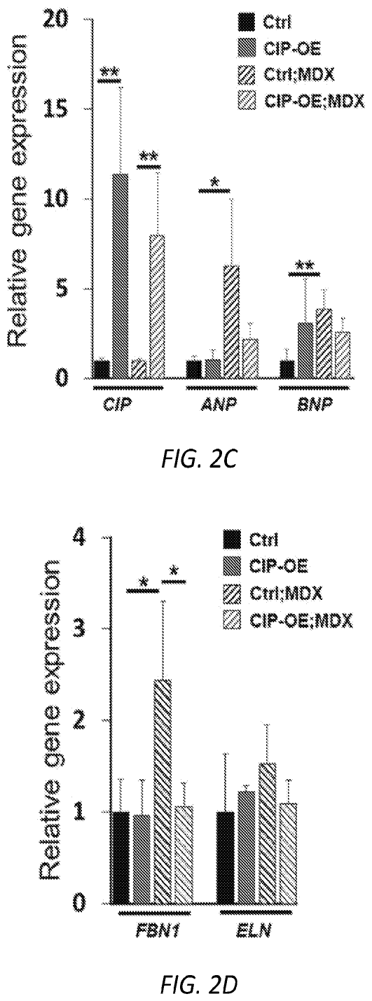

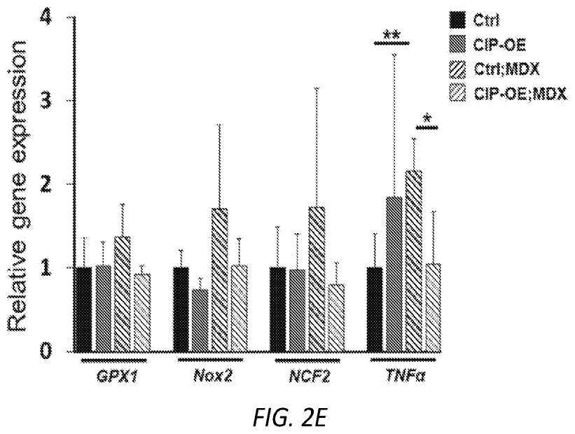

Next, we asked whether cardiac expression of CIP could protect dystrophic mice from developing cardiomyopathy. We bred CIP-overexpressing mice (CIP-OE), in which the CIP transgene is activated by MCK-Cre and overexpressed in heart and skeletal muscle, with mdx mice to create CIP-OE; mdx compound mice. CIP-OE mice appear normal, without detectable changes in cardiac function, under physiological conditions. Unlike human DMD patients, mdx mice develop mild, late onset cardiomyopathy^{21, 22}. Our 1-year-old mdx mice exhibit obvious dilated cardiomyopathy (enlarged left ventricular chamber, thinner ventricular wall, and enhanced cardiac fibrosis) with 100% penetrance, consistent with prior reports^{18, 21}. Cardiac function was preserved in 1-year-old CIP-OE; mdx mice compared to their control littermates (FIG. 2A), indicating that cardiac CIP overexpression blocks the development of cardiomyopathy in mdx mice. Histological analysis revealed dilated ventricular chambers and increased fibrosis in 1-year-old mdx mice, but no obvious cardiac dilation or fibrosis in 1-year-old CIP-OE; mdx mice (FIG. 2B). At 6 months of age, some of the mdx mice started to display cardiac dilation and increased fibrosis. In contrast, none of the CIP-OE; mdx mice exhibited pathological findings at this age. By 16 months of age, mdx mice displayed severe dilated cardiomyopathy with focal myocardial lesions and fibrosis, similar to what has been observed in DMD patients²³. In contrast, there was no ventricular dilation or obvious fibrosis in 16-month-old CIP-OE; mdx mice, indicating that CIP overexpression provides long-term protection from dystrophic cardiomyopathy. Consistent with the results of functional and cellular analysis, molecular marker gene studies revealed decreased expression of markers of cardiac disease (ANP, BNP) (FIG. 2C), fibrosis (FBN1) (FIG. 2D), and necrosis (TNFα) (FIG. 2E) in CIP-OE; mdx hearts. Together, these results demonstrate that overexpression of CIP in striated muscle blocks the progression of dystrophic cardiomyopathy in mdx mice.

### Molecular Signature of Dystrophic Cardiomyopathy

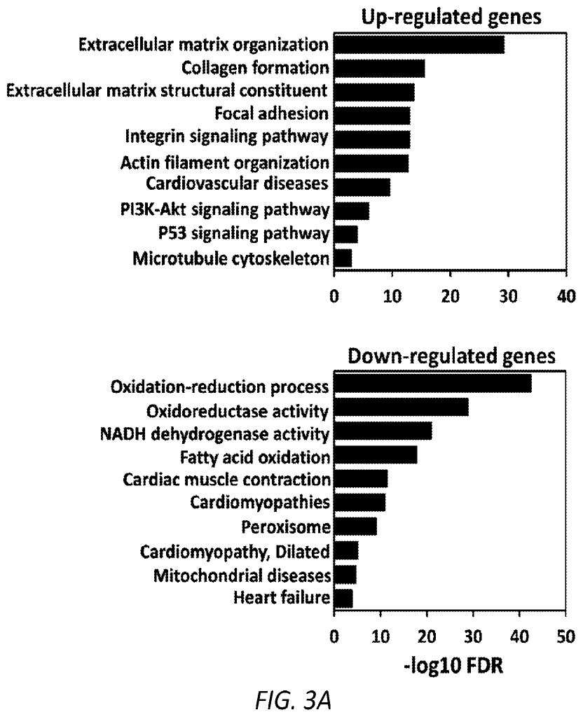

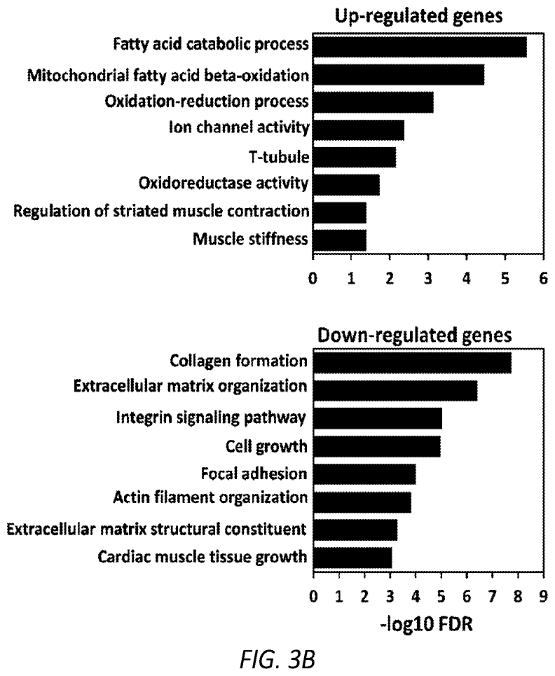

To define the molecular signature and mechanisms of dystrophic cardiomyopathy, we performed unbiased transcriptome analysis. RNA-seq of 3-month-old dKO and control mdx hearts identified 1,891 transcripts that were differentially expressed in CIP-KO; mdx hearts, including 855 down- and 1,036 up-regulated genes (P<0.01). Gene Ontology (GO) term analysis revealed that up-regulated genes were enriched for functional annotations related to extracellular matrix organization, collagen formation, and integrin signaling. Among the most up-regulated genes were genes associated with cardiomyopathy and heart failure (Myh7, NPPA, NPPB and Acta1) and cardiac fibrosis (collagen genes, Postn, ELN and tgfb2), consistent with our prior qRT-PCR analyses (FIGS. 1F, 1G). In contrast, down-regulated genes were enriched for functional annotations related to oxidation-reduction process, oxidoreductase activity and fatty acid oxidation (FIG. 3A). Several transcriptional factors and co-factors responsible for fatty acid metabolism, including Ppara, Ppargc1a, and Ppargc1b, were down-regulated, indicating that they may contribute to the pathology of dystrophic cardiomyopathy.

Next, we performed RNA-seq on 1-year-old CIP-OE; mdx and littermate control mice. A total of 791 genes, including 356 up- and 435 down-regulated, were dysregulated in CIP-OE; mdx hearts when compared with mdx controls (P<0.05). Intriguingly, GO term analysis demonstrated that the up-regulated genes were enriched for functional annotations related to fatty acid catabolic process, fatty acid beta-oxidation, and oxidoreductase activity, while the down-regulated genes were enriched for functional annotations related to collagen formation, extracellular matrix organization and integrin signaling pathway (FIG. 3B). These results suggest that cellular processes and molecular pathways for oxidation-reduction and fibrosis are key contributors in dystrophic cardiomyopathy.

The RNA-seq data from both young (3-month-old) and aged (1-year-old) mice with various genetic modifications to dystrophin and/or CIP, together with their distinctive pathological conditions, provide a unique opportunity to better understand the molecular signature of dystrophic cardiomyopathy progression. We performed an unbiased principal component analysis (PCA) using the whole transcriptome and found that 1-year-old CIP-OE; mdx hearts, which are phenotypically normal, are distant from 1-year-old mdx control hearts; instead, their transcriptome signature is similar to those of 3-month-old mdx controls and other littermate controls. Conversely, the transcriptome signature of 3-month-old CIP-KO; mdx hearts, which exhibit severe cardiomyopathy, is separate from that of 3-month-old mdx control hearts, instead grouping with 1-year-old mdx control hearts.

Next, we asked whether molecular signatures of dysregulated genes could be used to better define dystrophic cardiomyopathy phenotypes in these mice, in which dystrophic cardiomyopathy may or may not be present. Using unsupervised hierarchical clustering to analyze the 791 genes dysregulated in 1-year-old CIP-OE; mdx hearts as compared with age matched mdx controls, we found that all of the 1-year-old CIP-OE; mdx profiles were separate from 1-year-old mdx controls; instead, they were grouped with 3-month-old mdx controls and other littermate controls. These 791 genes grouped into five clusters. GO term analysis of these clusters showed that they were enriched for functional annotations related to Oxidoreductase, Membrane, Extracellular matrix, Calcium, and Glycoprotein. Similarly, unsupervised hierarchical clustering of the 3,236 genes dysregulated in 3 month-old CIP-KO; mdx hearts as compared with age matched mdx controls showed that that the 3-month-old CIP-KO; mdx hearts clustered with the 1-year-old mdx control hearts. Therefore, these analyses uncover an unique molecular signature of dystrophic cardiomyopathy and link the dysregulation of functional gene groups to the pathological condition of this disease.

### CIP Controls Dystrophic Cardiomyopathy Through the Oxidative Stress Pathway

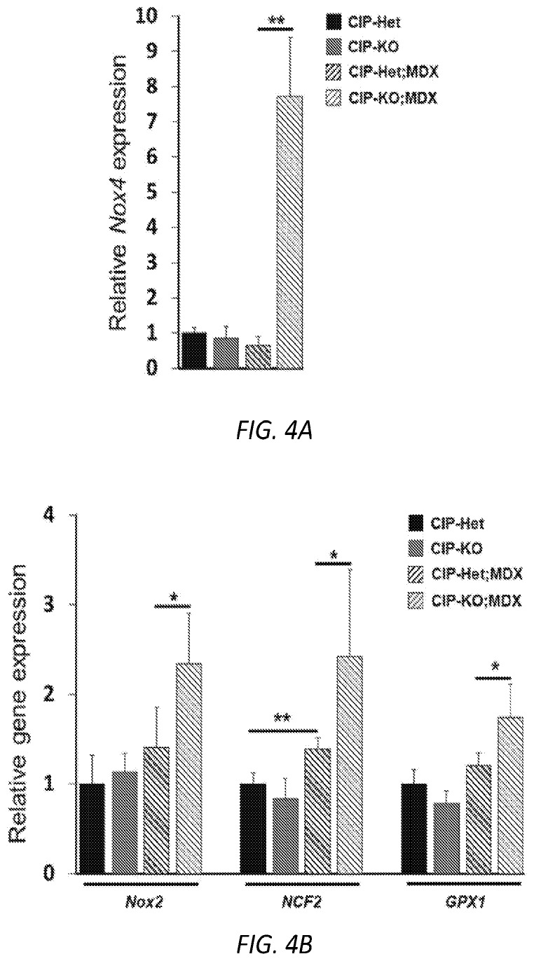

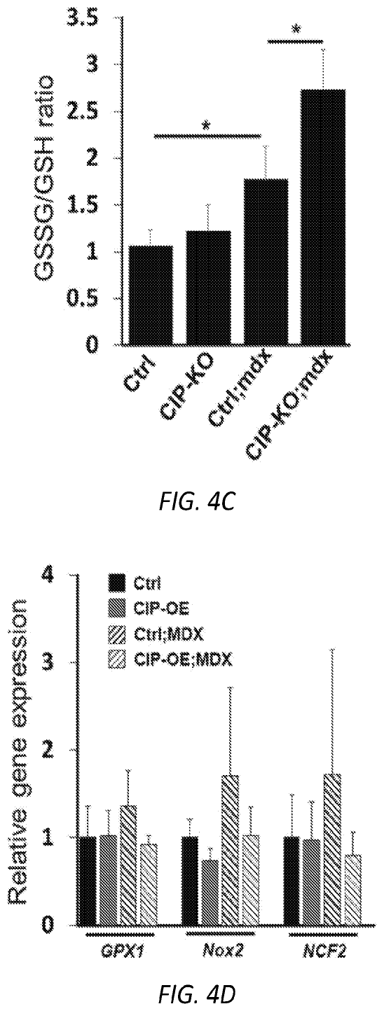

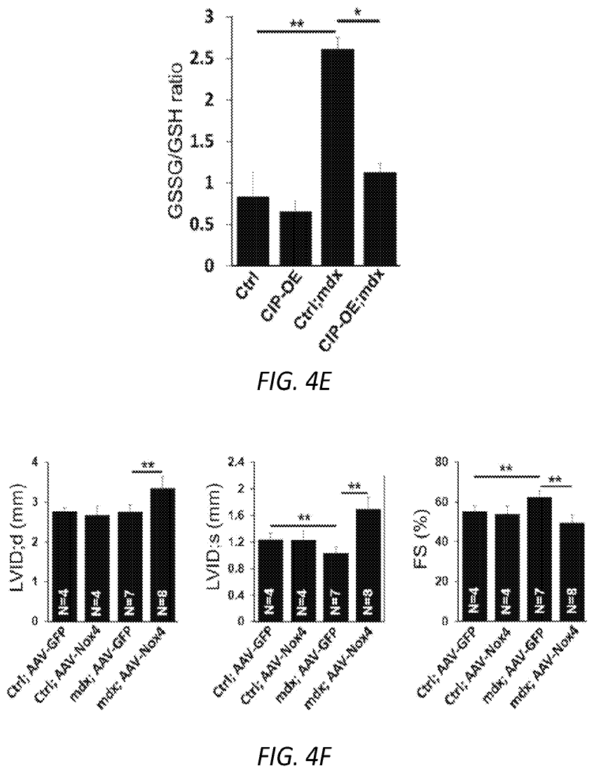

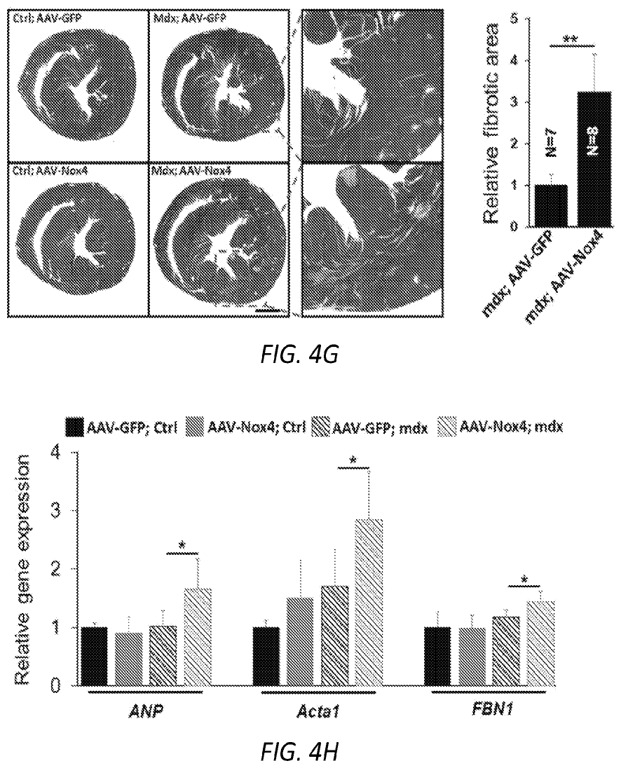

Among the most significantly dysregulated gene clusters in dystrophic cardiomyopathy were genes related to extracellular matrix (fibrosis) and the oxidative stress response. We reasoned that increased expression of fibrosis genes in dystrophic CIP-KO; mdx hearts and their reduced expression in healthy CIP-OE; mdx hearts may represent a consequence of cardiac remodeling. In contrast, we postulate that an increase in the oxidative stress response, as a result of reduced expression of oxidation-reduction process and oxidoreductase activity genes (FIG. 3B), could be a causative factor for dystrophic cardiomyopathy²⁴⁻²⁶. We confirmed that the expression of Nox4, an NADPH oxidase, is dramatically up-regulated in dKO hearts (FIG. 4A). Furthermore, the expression of other oxidases, including Nox2, NCF2, and GPX1, is also significantly increased in dKO hearts (FIG. 4B). To further confirm an increase in oxidative stress in dKO hearts, we measured the GSSG:GSH ratio, an indicator of oxidative stress²⁷, and found it dramatically higher in dKO hearts (FIG. 4C). Conversely, we found that the increased oxidase expression in 1-year-old mdx hearts is reduced by CIP overexpression in CIP-OE; mdx hearts (FIG. 4D). Similarly, we found that the GSSG:GSH ratio increases dramatically in 1-year-old mdx hearts, indicating a significant increase in oxidative stress in the aged mdx heart. This increased oxidative stress is rescued by the overexpression of CIP in CIP-OE; mdx hearts (FIG. 4E). Together, these data suggest that loss of function of CIP in the mdx heart elevates oxidative stress and accelerates cardiomyocyte death and cardiac fibrosis, whereas overexpression of CIP reduces oxidative stress to prevent the development of dystrophic cardiomyopathy.

The above data suggest that CIP modulates cardiac oxidative stress in dystrophic hearts mainly by regulating the expression and function of Nox4. We asked whether overexpression of Nox4 in the hearts of young mdx mice would be able to induce oxidative stress, leading to dystrophic cardiomyopathy, similar to what we saw in the CIP-KO; mdx dKO mice. We achieved cardiac-specific Nox4 overexpression in mdx and control mice using an Adeno Associated Virus 9 (AAV9) delivery system (Suppl. online FIG. 2). As expected, AAV9-mediated Nox4 overexpression in the heart of mdx mice accelerates cardiac remodeling, as evidenced by an increase in ventricle chamber dimension (LVID) and a decrease in cardiac contraction (FS %) (FIG. 4F). Accordingly, there is a substantial increase in cardiac fibrosis (FIG. 4G). Molecular marker analysis revealed increased expression of ANP and Acta1, both markers of cardiomyopathy, and fibronectin (FBN1), which marks fibrosis (FIG. 4H). In contrast, increased Nox4 expression does not alter cardiac function or remodeling in control animals (FIGS. 4F-4H). Together, these data indicate that Nox4 accelerates the development of cardiomyopathy in the absence of dystrophin, identifying Nox4 as a critical mediator of dystrophic cardiomyopathy.

CIP Interacts with Dystrophin and Calcineurin (CnA) to Regulate CnA-NFAT-Nox4 Signaling and Dystrophic Cardiomyopathy

In order to better understanding the molecular mechanism by which CIP regulates oxidative stress during dystrophic cardiomyopathy, we undertook an unbiased approach to identify CIP-interacting proteins in the heart. We used GST-CIP to pull down interacting proteins from heart extracts. Subsequent mass spectrometry identified multiple candidate CIP-interacting proteins, including LMO7, HSPA9, α-Actinin, β-Tubulin and calcineurin (CnA). Their interaction with CIP was confirmed by co-immunoprecipitation (co-IP) assays (Suppl. online FIG. 3). Next, we examined the cellular location of CIP protein in cardiomyocytes. CIP protein is predominantly localized to the cardiomyocyte cell surface, where it co-localizes with dystrophin (FIG. 5A). A direct interaction between CIP and dystrophin was confirmed by co-IP assays (FIG. 5B).

CnA is a protein phosphatase that regulates cardiac remodeling^{17, 28-30}. We have previously reported that genetic and functional interactions between CnA and CIP modulate cardiac remodeling¹⁴. Interestingly, CnA has been linked to the expression and function of the Nox4 gene in the kidney³¹. We therefore hypothesized that the interplay of CIP, CnA, and dystrophin modulates downstream oxidative stress and thereby critically regulates the development of dystrophic cardiomyopathy. Co-IP assay showed that CIP interacts with CnA but not CnB or CIB1, a scaffold protein that interacts with CnB and the sarcolemma³² (FIG. 5C). After activation, CnA dephosphorylates nuclear factor of activated T cells (NFAT) to facilitate its nuclear translocation and the activation of downstream hypertrophic genes¹⁷(6). Using an EGFP-tagged NFAT protein, we found that overexpression of CIP alone does not affect the subcellular distribution of NFAT. However, in the presence of activated CnA, CIP dramatically reduces CnA-activated NFAT nuclear translocation (FIG. 5D). Quantitative analysis further supports this conclusion (FIG. 5E). As a consequence, overexpression of CIP significantly represses CnA-NFAT-activated NFAT-Luciferase activity in a dose-dependent manner, whereas CIP by itself does not affect NFAT-Luciferase activity (FIG. 5F).

A previous study showed that the CnA-NFAT signaling cascade regulates the expression and function of oxidases Nox4 and Nox2 in the kidney³¹. We asked whether these oxidases are similarly regulated by CIP-CnA-NFAT signaling in cardiomyocytes. We overexpressed or knocked down CIP in neonatal rat ventricular myocytes (NRVM). Cells were treated with phenylephrine to activate CnA-NFAT signaling and induce cardiomyocyte hypertrophy^{33, 34}. Knock down of CIP induces Nox4 expression, while CIP overexpression results in a reduced Nox4 level (FIG. 5G).

We examined the regulation of Nox4 and oxidative stress by CIP and CnA in vivo. AAV9-mediated cardiac overexpression of CIP markedly represses the level of Nox4, which is dramatically induced in the hearts of CnA transgenic mice (FIG. 5H). As a result, the GSSG:GSH ratio in CnA transgenic heart is decreased when CIP is overexpressed, indicating that CIP is sufficient to lower oxidative stress in CnA transgenic hearts (FIG. 5I). Together, these data suggest that CIP regulates oxidative stress in cardiomyocytes during cardiac remodeling through the CnA-NFAT-Nox4 signaling cascade.

### Example 2. CIP, a Cardioprotective Factor, Inhibits the Transition from Cardiac Hypertrophy to Heart Failure

Heart failure is currently incurable and is characterized by the failure of the heart to pump enough blood to meet the body's needs and affects more than 100 million people globally. Little is known about how the transition from cardiac hypertrophy to heart failure is regulated. Previously, we identified a cardiomyocyte-enriched gene, CIP, which regulates cardiac homeostasis under pathological conditions. Here, we demonstrate that the promotor of the CIP gene contains binding sites for GATA4 and the expression of CIP is regulated by this cardiac transcription factor. We also determined that both CIP mRNA and protein decrease in diseased human hearts. In a mouse model of heart failure, induced cardiac-specific overexpression of CIP after the onset of hypertrophy was sufficient to prevent progression to heart failure. Transcriptome analyses with RNA-seq revealed that IGF, mTORC2, and TGFβ signaling pathways meditate the inhibitory function of CIP on pathological cardiac remodeling. Our study reveals the mechanism by which CIP gene expression is controlled in cardiomyocytes and underscores the clinical relevance of CIP in heart disease. More importantly, our investigation suggests that CIP is a key regulator of the transition from cardiac hypertrophy to heart failure. These results indicate the therapeutic potential of CIP for treating the failing heart.

### Methods

The following materials and methods were used in this Example.

Human samples. Left ventricular (LV) tissues were taken from patients with terminal-stage heart failure indicated for heart transplantation performed in the First Affiliated Hospital, Sun Yat-sen University. In brief, the patient's heart was removed at the time of transplantation and LV tissue was subsequently dissected and snap-frozen. We used LV samples from not implanted healthy hearts to serve as controls. All the procedures followed the protocol approved by the First Affiliated Hospital, Sun Yat-sen University, Guangzhou, China.
Mice. CIP-KI-flox mice were generated in previous study¹⁴. CIP-KI-flox mice, which have a Rosa-CIP allele (the stop codon is present and floxed) were bled with aMHC-Mer-Cre-Mer mice to obtain CIP-OE (CIP-KI-flox; aMHC-Mer-Cre-Mer) mice. Tamoxifen was administrated through intraperitoneal injection to activate the expression of Cre recombinase and the excision of the stop codon for the ectopic expression of CIP transgene in the heart in CIP-OE mice. CIP-KI-flox littermates were used as controls.
Measurement of cardiac function by echocardiography. Echocardiographic measurements were performed on mice using a Visual Sonics Vevo® 2100 Imaging System (Visual Sonics, Toronto, Canada) with a 40 MHz MicroScan transducer (model MS-550D). Mice were anesthetized with isoflurane (2.5% isoflurane for induction and 0.1% for maintenance). Heart rate and LV dimensions, including diastolic and systolic wall thicknesses, LV end-diastolic and end-systolic chamber dimensions were measured from 2-D short-axis under M-mode tracings at the level of the papillary muscle. LV mass and functional parameters such as percentage of fractional shortening (FS %) and ejection fraction (EF %) were calculated using the above primary measurements and accompanying software.
Transverse aortic constriction operation. Mice were anesthetized with isoflurane (3-4% isoflurane for induction, 1-2% isoflurane for maintenance). The chest was shaved and cleaned with alcohol. A suture was placed around the front upper incisors and pulled taut so that the neck was slightly extended. The tongue was retracted and held with forceps, and a 20-G catheter was inserted into the trachea. The catheter was then attached to the mouse ventilator via a Y-shaped connector. Ventilation was performed with a tidal volume of 220-240 μl for a 25-30 g mouse and a respiratory rate of 130-140 breaths per min. 100% oxygen was provided to the inflow of the ventilator. The chest was opened through a left 2^{nd} intercostal thoracotomy. The 26-G needle without its sharp tip was put on the ascending aorta. They were tightly ligated together using 7-0 Nylon suture (Ethicon) at the position between brachiocephalic artery and left common carotid artery, and the 26-G needle was removed immediately after ligation. In the sham operation, no ligation was performed. Isoflurane was stopped, and the lungs were slightly overinflated to assist in removal of air in the pleural cavity. Dissected intercostal space and chest skin were closed using 6-0 silk suture (Ethicon). All manipulations were performed by an operator without knowledge of genotype

Haematoxylin and eosin staining and fast green/Sirius red collagen staining. Mouse heart tissues were dissected out, rinsed with PBS and fixed in 4% paraformaldehyde (pH 8.0) overnight. After dehydration through a series of ethanol baths, samples were embedded in paraffin wax according to standard laboratory procedures. Sections of 5 μm were stained with haematoxylin and eosin (H&E) for routine histological examination with light microscope. For Sirius red/fast green collagen staining sections were fixed with pre-warmed Bouins' solution, 55° C. for 1 hour then washed in running water. Sections were stained in 0.1% fast green solution for 10 minutes then washed with 1% Acetic acid for 2 minutes. After rinsing in tape water, sections were stained in 0.1% Sirius resolution for 30 minutes. After staining, sections were dehydrated and cleared with Xylene. The images were examined with light scope and quantified with ImageJ software.

Quantitative RT-PCR and Western blot analysis. Total RNAs were isolated using Trizol Reagent (Life technologies) from cells and tissue samples. For Quantitative RT-PCR, 2.0 μg RNA samples were reverse-transcribed to cDNA by using random hexamers and MMLV reverse transcriptase (Life technologies) in 20 μl reaction system. In each analysis, 0.1 μl cDNA pool was used for quantitative PCR. The relative expression of interested genes is normalized to the expression of ACTB or PRKG1. Primers used in this study are listed in the table below. For Western blot analyses, tissue homogenate were cleared by 10,000×g centrifugation for 10 min. Samples were subsequently analyzed by SDS/PAGE and transferred to PVDF membranes that were incubated with 5% non-fat dry milk in TBST and Anti-CIP (1:2000, 21st Century Biochemical) or Anti-GAPDH (1:5000, Proteintech) overnight at 4° C. and then washed three times with TBST buffer before adding IgG secondary antibody. Specific protein bands were visualized through chemiluminescent detection.

PRIMERS

| | | SEQ | | SEQ |
|---|---|---|---|---|
| Gene | | ID | | ID |
| name | Forward primer (5′→3′) | NO: | Reverse primer (5′→3′) | NO: |
| NPPA | CACAGATCTGATGGATTTCAAGA | 1. | CCTCATCTTCTACCGGCATC | 2. |
| NPPB | GTCAGTCGTTTGGGCTGTAAC | 3. | AGACCCAGGCAGAGTCAGAA | 4. |
| FBN1 | CCTTCCTGTGGCTCCAGAT | 5. | GCTGCCCCCATTCATACA | 6. |
| Acta1 | GCCCATCTATGAGGGCTATG | 7. | AATCTCACGTTCAGCTGTGG | 8. |
| Tgfb1 | AAGACTTCACCCCAAAGCTG | 9. | GAGAGAGGGTCCTGGGATG | 10. |
| Des | GCGTGACAACCTGATAGACG | 11. | TGGATTTCCTCCTGTAGTTTGG | 12. |
| Bax | GTGAGCGGCTGCTTGTCT | 13. | GTGGGGGTCCCGAAGTAG | 14. |
| Cebpa | CGCTGGTGATCAAACAAGAG | 15. | GGTGGCTGGTAGGGGAAG | 16. |
| Cebpb | TGATGCAATCCGGATCAA | 17. | CACGTGTGTTGCGTCAGTC | 18. |
| Cox6a2 | GAGCGCCCAGAGTTCATC | 19. | TGTGGAAAAGCGTGTGGTT | 20. |
| Cox8b | AGCCAAAACTCCCACTTCC | 21. | GAACCATGAAGCCAACGAC | 22. |
| Rictor | GGTGATAACTACGTTCGTCGC | 23. | AAAGGTGTACGGGCAGGTAG | 24. |
| Bbc3 | TTCTCCGGAGTGTTCATGC | 25. | TACAGCGGAGGGCATCAG | 26. |
| Ndufa1 | TGATGGAACGCGATAGACG | 27. | GCCAGGAAAATGCTTCCTTA | 28. |
| Ndufb9 | TTTCCAAGAGAGAGCAGTGGA | 29. | CTCCTGCAGCTGCTTAACCT | 30. |
| Uqcr10 | ACTTCCACCTTTGCCCTCAC | 31. | TCCACAGTTTCCCCTCGTTG | 32. |
| Uqcr11 | CCACAGGCCTCGATGGTA | 33. | GCAGCCCTAGTGTCTGTCAA | 34. |
| Cox5a | TTAAATGAATTGGGAATCTCCAC | 35. | GTCCTTAGGAAGCCCATCG | 36. |
| Sod3 | CTCTTGGGAGAGCCTGACA | 37. | GCCAGTAGCAAGCCGTAGAA | 38. |
| Junb | CCACGGAGGGAGAGAAAATC | 39. | AGTTGGCAGCTGTGCGTAA | 40. |
| Fgfr1 | GACCTACGTTCAAGCAGTTGG | 41. | TCCAGCGGTATGGACAGG | 42. |
| Smad7 | ACCCCCATCACCTTAGTCG | 43. | GAAAATCCATTGGGTATCTGGA | 44. |
| Rhoc | AAGGACCTGAGGCAAGATGA | 45. | AAGGCACTGATCCTGTTTGC | 46. |
| Pdgfb | CGAGGGAGGAGGAGCCTA | 47. | GTCTTGCACTCGGCGATTA | 48. |
| Igfbp7 | TGCCCTCCATGAAATACCAC | 49. | GGCTGTCTGAGAGCACCTTT | 50. |
| CIP | TAGCTACTCGGCCCAAGTCT | 51. | ATCCCATGAGGAATTTCAGG | 52. |
| PRKG1 | TCCAACATTCCAGAGCCTTC | 53. | TTTTCATAGTGGGTCTCTTCGAG | 54. |
| β-actin | GATCTGGCACCACACCTTCT | 55. | GGGGTGTTGAAGGTCTCAAA | 56. |

Constructs, cell culture, and luciferase reporter assays. HEK293T cells were cultured in DMEM supplemented with 10% FBS in a 5% CO2 atmosphere at 37° C. Wildtype, mutant or truncated CIP promoter sequences were cloned into multiple cloning sites of the pGL3-Basic vector (Promega) to generate CIP-Luc reporters used in this study. An indicated combination of CIP-Luc reporter, pRL Renilla reporter (internal control) and GATA4 construct were transfected into HEK293T cells with PEI reagents. 48 hours after transfection, cell extracts were prepared and luciferase activity was determined. For dual-luciferase assay, normalized firefly luciferase expression from triplicate samples in 12-well plates relative to renilla luciferase expression was calculated.
Mouse heart RNA-seq data analyses. Total RNAs from mouse heart were used to perform RNA-seq in BGI Genomics (Wuhan, China). RNA-seq reads were mapped to mouse genome mm10 by STAR and reads counts were calculated with FeatureCounts. Expression analysis was run in RStudio. DESeq2 was employed to perform statistical analysis of differential gene expression. An adjusted P value of 0.05 were used as cutoff to identify differentially regulated genes. Volcano plot were performed with the ggplot2 library. Hierarchical clustering heatmap was made with the pheatmap library.
Human heart RNA-seq data collection and analyses. Human diseased heart RNA-seq data, including GSE57344, GSE71613, GSE116250, GSE46224, GSE108157, GSE55296, GSE120836, GSE130036, were downloaded from NCBI database. Gene-level quantification were calculated by featureCounts-v1.6.3. To perform strand-specific reads counting, the strand type (nonstrand, stranded, reversely stranded) of each sample was inferred from sorted bam file using infer_expriment.py (3.0.0). Then we can provide featureCounts with strand type information to calculate read counts of every gene in each sample and merged the quantification results together to make an expression matrix for differential gene expression analysis. Differential gene expression analysis was performed using DESeq2-1.24.0. The design matrix in DESeq2 model was written as “˜series+gender+phenotype” to adjust the differences between data series and gender. Only differential expressed genes with FDR<0.05 and log 2FoldChange>0.25 identified by DESeq2 were kept. Then, we applied the classic weighted correlation network analysis (WGCNA) algorithm for co-expression analysis. The R implementation of WGCNA (version: 1.68) was used in our study.
Statistics. Values are reported as means±SEM unless indicated otherwise. Statistical significance was determined with ANOVA. For multiple group comparisons, a post-hoc Tukey's test was performed when ANOVA reached significance. Values of P<0.05 were considered statistically significant.

### Results.

### Cardiac Transcription Factor GATA4 Binds to the Promotor of CIP and Regulates its Expression

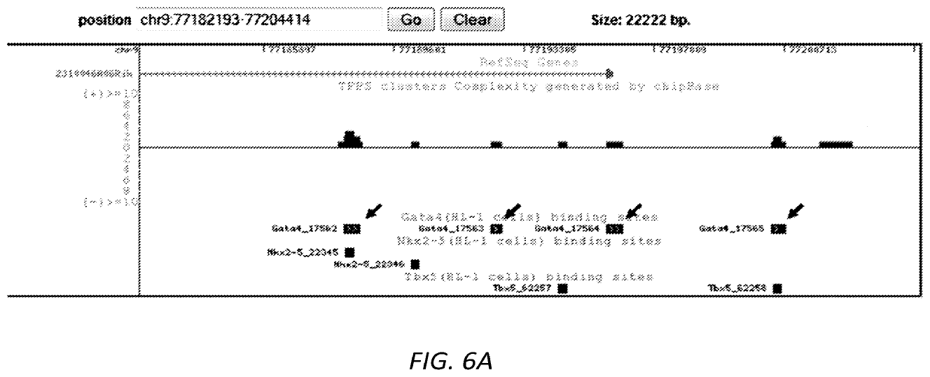

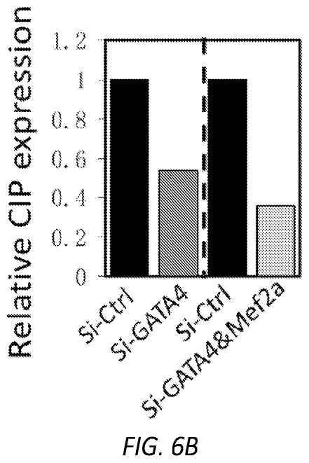

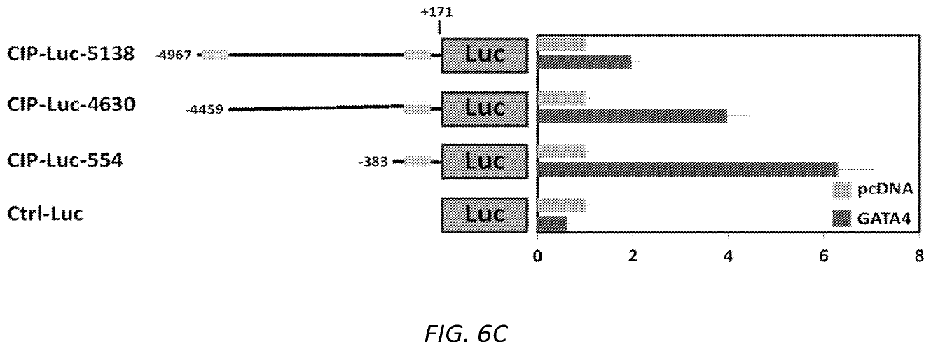

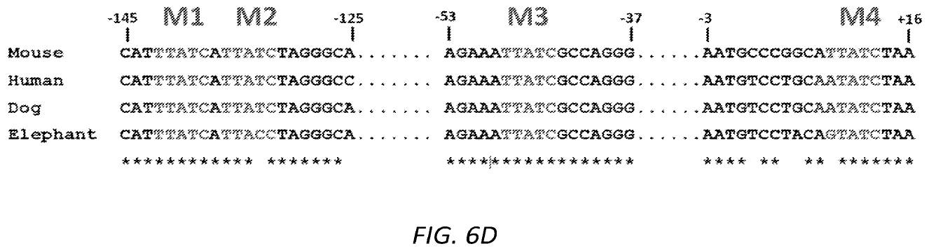

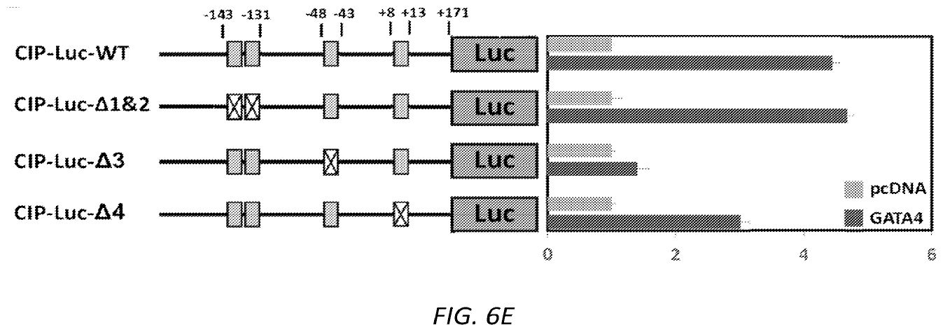

CIP (Cardiac ISL1-interacting Protein)¹⁴ or MLIP (Muscle-enriched A-type Lamin-Interacting Protein)¹² has been previously identified as a striated muscle-enriched gene. In the heart, CIP is predominantly expressed in cardiomyocytes; however, the transcription factors that control the expression of CIP in these cells have yet to be thoroughly investigated. Genome-wide binding sites for multiple cardiac transcriptional factors, including GATA4, Tbx5, Nkx2-5, Mef2A, and SRF, have been carefully investigated⁵¹. GATA4, Tbx5 and Nkx2-5 were found to bind to the promotor region of CIP (FIG. 6A). Knock-down of GATA4, or a combination of GATA4 and Mef2a, decreased expression of CIP in cardiac cells (FIG. 6B), indicating GATA4 is a key regulator of CIP. To determine whether CIP is directly controlled by GATA4, we generated luciferase reporter constructs using different lengths of the CIP promotor and first exon, which contained GATA4-binding sequences as detected by CHIP-seq⁵¹. In transient cell-based luciferase assays, all CIP-Luc reporters were responsive to GATA4 transactivation. The 554 bp CIP-Luc reporter, containing a proximal GATA4 binding sequence, was the most responsive, indicating a functional GATA4 enhancer was located in this sequence (FIG. 6C). Four putative GATA4 binding motifs, which were evolutionarily conserved, were found within the 554 bp CIP promotor region (FIG. 6D). To determine the functional binding motifs, we generated three mutant 554 bp CIP-Luc reporters that contain motif 1&2 mutants, a motif 3 mutant, and a motif 4 mutant, respectively. Luciferase assays showed mutation of motif 3 and motif 4, but not motif 1&2, decreased the response of the CIP-Luc reporter to GATA4 transactivation (FIG. 6E), suggesting motif 3 and motif 4 are functional GATA4 binding motifs. Therefore, our data demonstrated that GATA4 is a key transcription factor that directly regulates the expression of CIP in cardiomyocytes.

Decreased Expression of CIP Correlated with Dysregulated Oxidative Phosphorylation (OXPHOS) in Multiple Human Heart Diseases

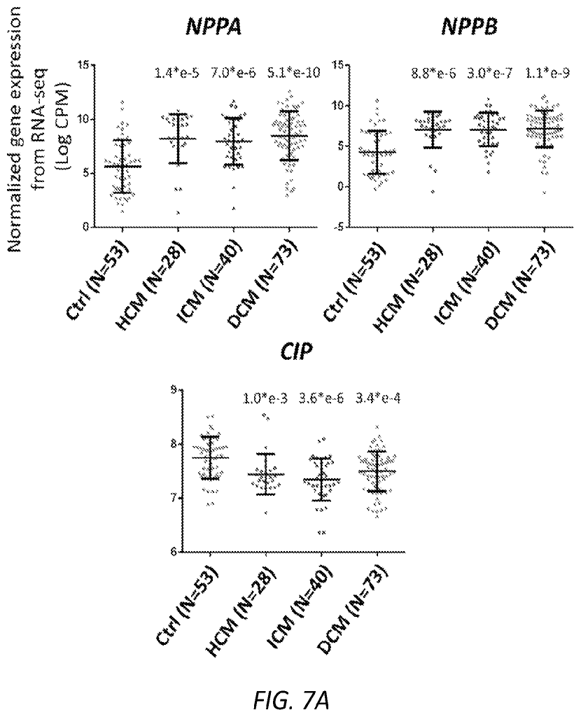

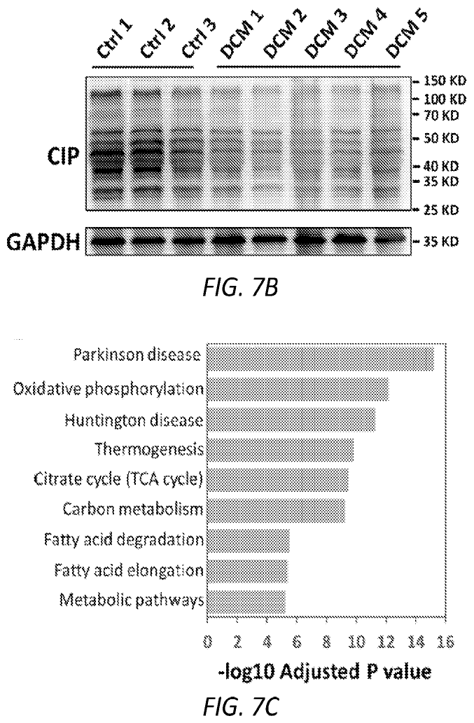

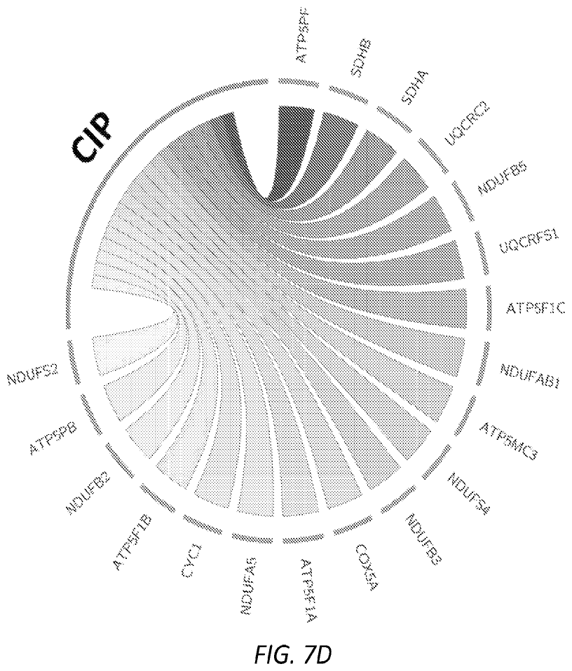

To further investigate the relevance of CIP to human cardiac diseases, we collected RNA-seq data from 194 human hearts using a public database, including 53 non-failing heart samples (NF), 28 hypertrophic cardiomyopathy samples (HCM), ischemic cardiomyopathy samples (ICM), and 73 dilated cardiomyopathy samples (DCM). After normalizing the data, we found the expression of CIP was significantly decreased in all diseased samples, while the cardiac disease markers, NPPA and NPPB, were dramatically upregulated (FIG. 7A). Furthermore, the downregulation of CIP in human DCM hearts was confirmed by Western blot analysis (FIG. 7B). Next, we performed co-expression analyses to explore the potential CIP gene regulatory network. Genes from the human genome were divided into multiple modules based on the Spearman correlation coefficient score within the module. The module containing CIP was further subjected to KEGG pathway analysis. Pathways related to “Parkinson disease”, “oxidative phosphorylation”, “Huntington disease” and “metabolic pathways” were on the top of the list (FIG. 7C). Given that the heart was the focus of our study, our data indicated that CIP is involved in the cardiac regulation of OXPHOS. OXPHOS-related genes with the strongest correlation coefficient were further shown in a Circos plot (FIG. 7D). Together, our data from genome-wide analyses of human heart diseases suggested that CIP plays an important role in human heart disease by regulating oxidative phosphorylation in cardiomyocytes.

Cardiac Overexpression of CIP Protects the Diseased Heart from Transitioning Toward Heart Failure

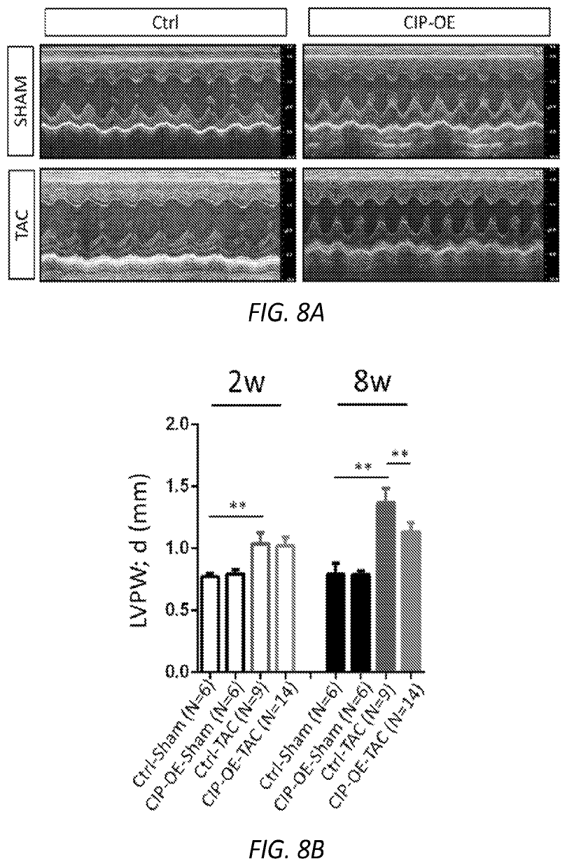

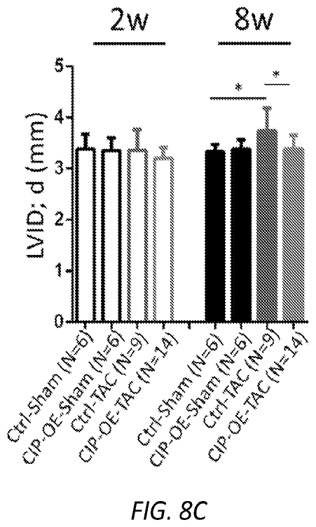

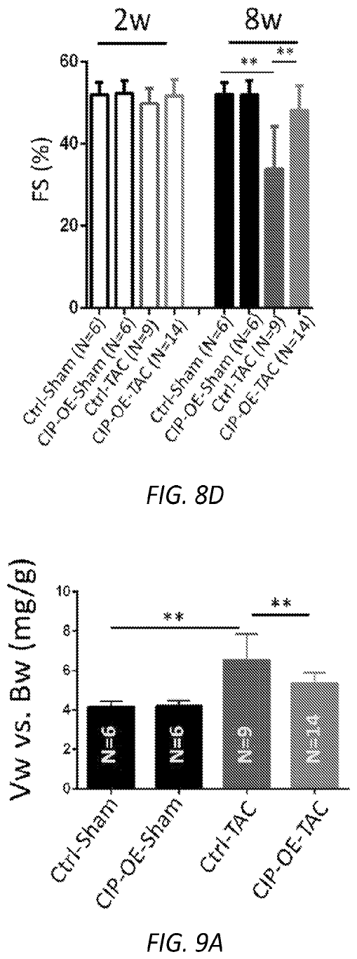

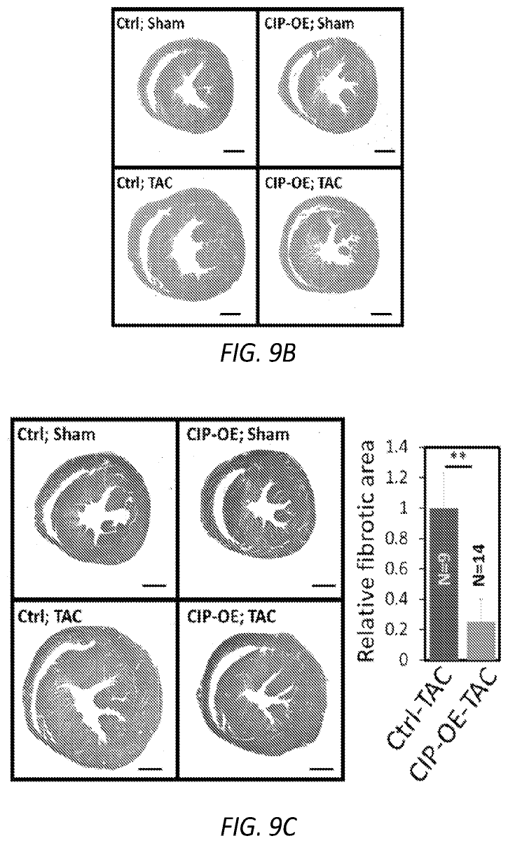

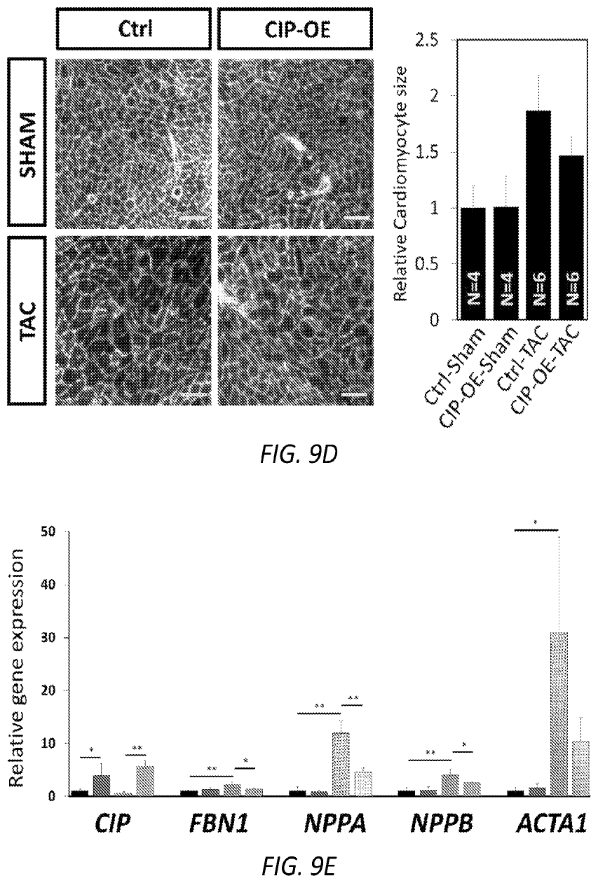

In a previous study, we reported that overexpression of CIP in the heart before cardiac stress inhibited maladaptive remodeling¹⁴. In order to further test the effect of CIP in treating the progression of adverse cardiac remodeling, we performed transverse aortic constriction (TAC) surgery, which induces ventricular pressure overload, on an inducible cardiac-specific CIP overexpressing mice (Rosa26-CIP-flox; Myh6-MerCreMer, CIP-OE mice). Mice were administered Tamoxifen to induce cardiac-specific overexpression of CIP 2 weeks after TAC surgery. Cardiac remodeling was confirmed 2 weeks after TAC by echocardiography of the left ventricular posterior wall (LVPW; FIG. 8A, 8B). Cardiac parameters were measured at 2 weeks and 8 weeks post-surgery by echocardiography. Compared to the Sham-operated group, the ventricular wall became thicker and the ventricular chamber was dilated in control mice (FIG. 8A, 8C). As expected, cardiac function as measured by fraction shortening (FS) decreased significantly, indicating the progression to heart failure (FIG. 8A, 8D). In contrast, overexpression of CIP during hypertrophy significantly repressed the thickening of ventricular wall as well as chamber dilation. More importantly, cardiac function was preserved by CIP overexpression during the prolonged period of stress (FIG. 8A-D). Mice were sacrificed at 8 weeks post-surgery for cardiac sample collection. Consistent with echocardiographic data, significant adverse cardiac remodeling was induced by pressure overload in control mice, as indicated by the ratio of ventricular weight to body weight (Vw vs. Bw) and histological examination (FIG. 9A, 4B). Overexpression of cardiac CIP led to a smaller heart under pressure overload. Consistently, the size of cardiomyocytes, indicated by their cross-sectional area as determined by Wheat Germ Agglutinin staining, was significantly larger in the control group after TAC compared to the CIP overexpressing mice (FIG. 9C). Because heart failure is often accompanied with increased cardiac fibrosis, we performed Sirius Red/Fast Green staining of hearts, which showed less fibrosis in the CIP-OE hearts under cardiac stress compared to the control group (FIG. 9D). We further examined molecular markers of cardiac disease and fibrosis, including NPPA, NPPB, ACTA1, and FBN1. The expression of these genes by cardiac stress was significantly repressed by CIP overexpression (FIG. 9E). Together, these data demonstrated cardiac overexpression of CIP after initiation of pathological remodeling inhibited disease progression and protected the heart from failure.

The Protective Function of CIP is Mediated by IGF and mTORC2 Signaling Pathways

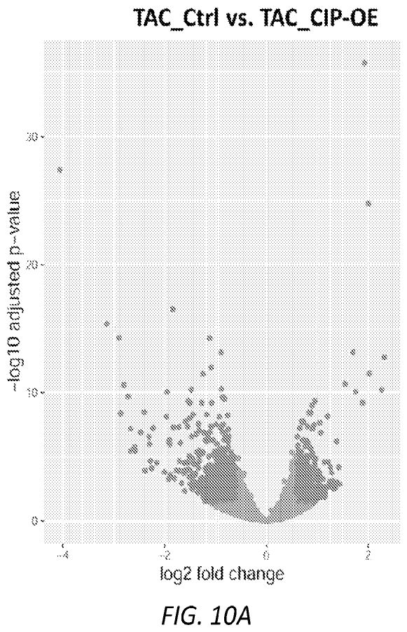

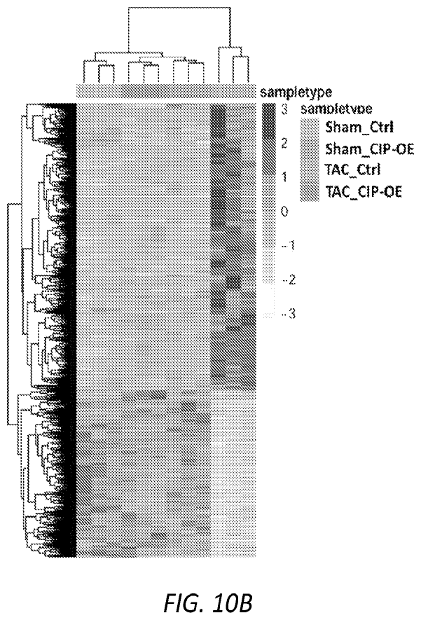

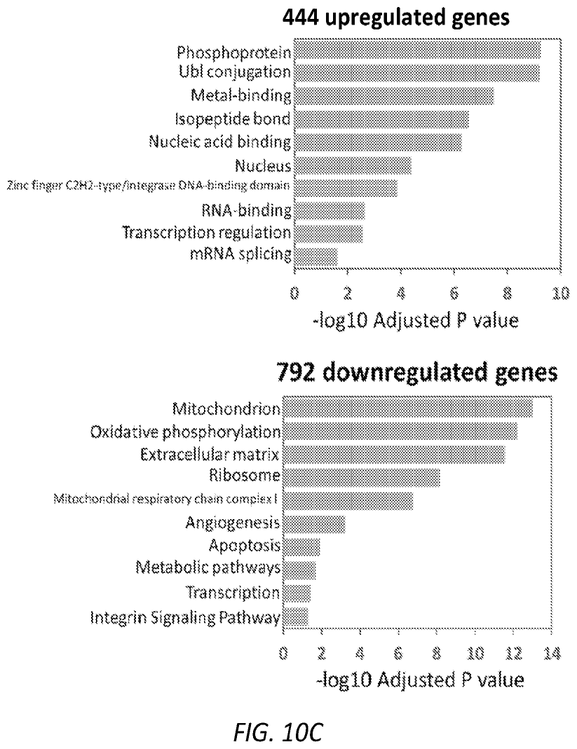

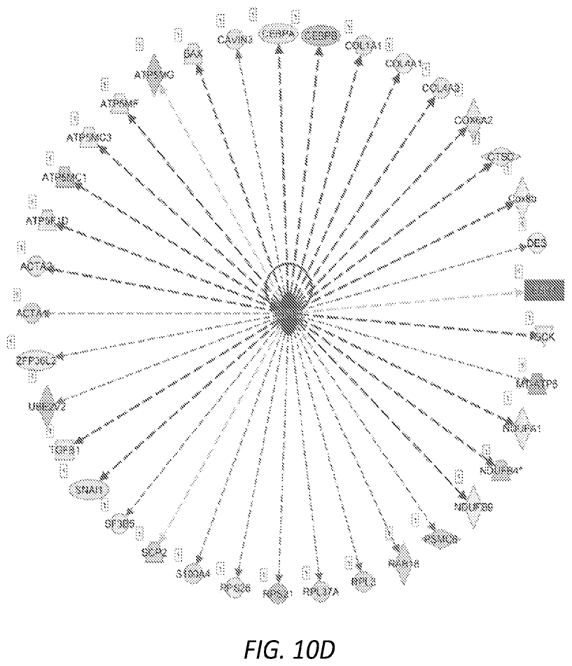

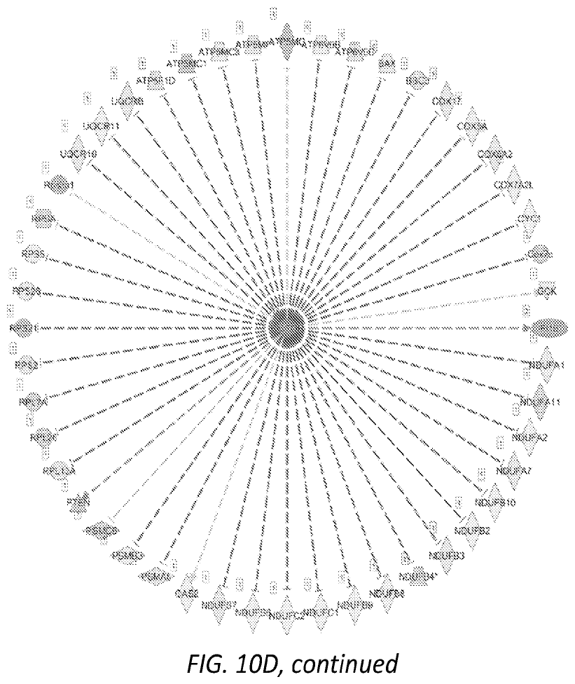

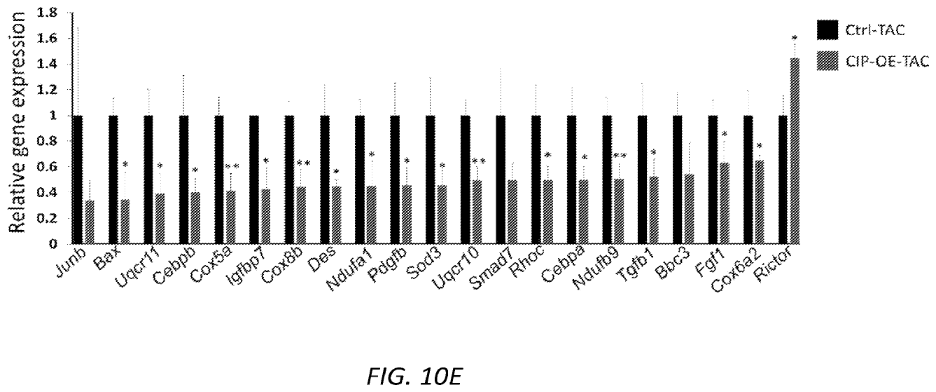

To investigate the potential mechanism of protection by CIP, we carried out unbiased transcriptome profiling with hearts of CIP-OE mice at 8 weeks post-surgery using RNA-seq. In total, 444 genes, including CIP, were significantly up-regulated, while 792 gene were significantly down-regulated in CIP-KI hearts (fold change>1.5 and adjust p<0.05) (FIG. 10A). A hierarchical clustered heatmap revealed that the dysregulation of genes induced by TAC surgery in the control group was essentially rescued by CIP overexpression in stressed hearts (FIG. 10B). To further characterize this subset of genes, 1236 dysregulated genes were subjected to GO term analyses. Consistent with what we had found in human diseased hearts, genes related to “Mitochondrion” and “Oxidative phosphorylation” were enriched in the down-regulated genes (FIG. 10C). To gain more insight of the gene regulation network, we subsequently searched for upstream regulators of these dysregulated genes with Ingenuity Pathway Analysis (IPA). Several regulators, including IGF1R (p=7.27e-8) (FIG. 10D), Rictor (a core component of mTORC2, p=6.25e-14) (FIG. 10E), and TGFB1 (p=4.76e-7), topped the list. It is worth noting that IGF1R was reported to modulate the activity of FoxO1 through regulation of AKT, which is consistent with our previous report that showed CIP regulated pathological cardiac remodeling through the FoxO1/CnA signaling cascades¹⁵. Dysregulation of downstream targets for IGF1R from IPA analysis, including TGFB1, BAX, CEBRA, CEBRB, COX6A2, COX8B, DES, NDUFA1, and NDUFB9, as well as several downstream targets for Rictor and TGFB1 were confirmed by qRT-PCR (FIG. 10F). All of these data suggest that the inhibitory function of CIP on cardiac remodeling was mediated, at least in part, by IGF and mTORC2 signaling pathways.

### Example 3. Control of Nuclear Positioning by Skeletal Muscle CIP

The appropriate arrangement of myonuclei within skeletal muscle myofibers is of critical importance for normal muscle function, and improper myonuclear localization has been linked to a variety of skeletal muscle diseases, such as centronuclear myopathy and muscular dystrophies. However, the molecules that govern myonuclear positioning remain elusive. Here we report that muscle-specific CIP (sk-CIP) is a novel regulator of nuclear positioning. Genetic deletion of sk-CIP in mice results in misalignment of myonuclei along the myofibers and at specialized structures such as the neuromuscular junctions (NMJs) and myotendinous junctions (MTJs) in vivo, impairing myonuclear positioning after muscle regeneration, leading to severe muscle dystrophy in mdx mice, a mouse model of Duchenne muscular dystrophy. sk-CIP is localized to the centrosome in myoblasts and relocates to the outer nuclear envelope in myotubes upon differentiation. Mechanistically, we found that sk-CIP directly interacts with the Linker of Nucleoskeleton and Cytoskeleton (LINC) complex and the centriole Microtubule Organizing Center (MTOC) proteins to control myonuclear positioning and alignment. These findings identify sk-CIP as a long-sought muscle-specific anchoring protein that controls myonuclear position. sk-CIP could be a therapeutic target for muscle related diseases such as muscular dystrophy.

### CIP Regulates Myonuclear Position During Myogenic Differentiation

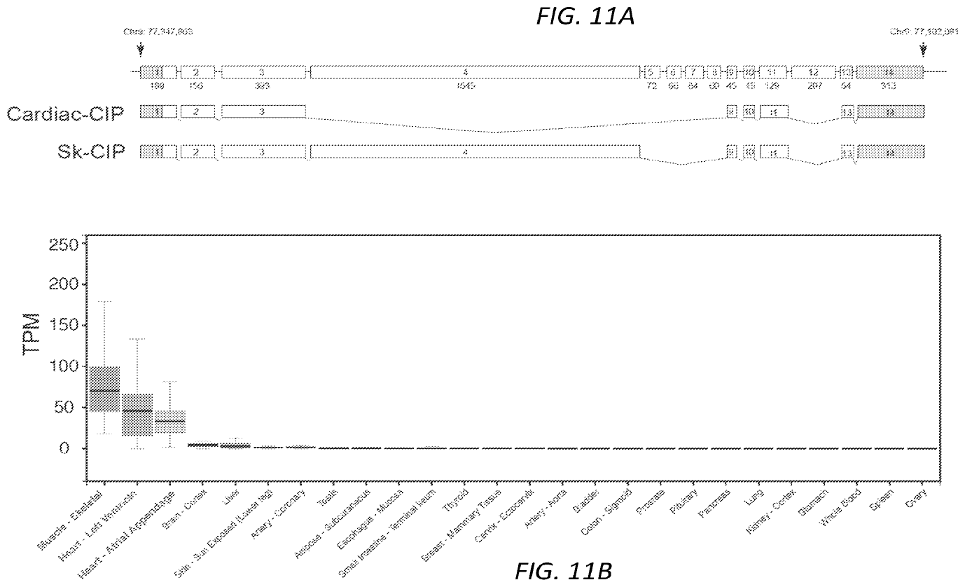

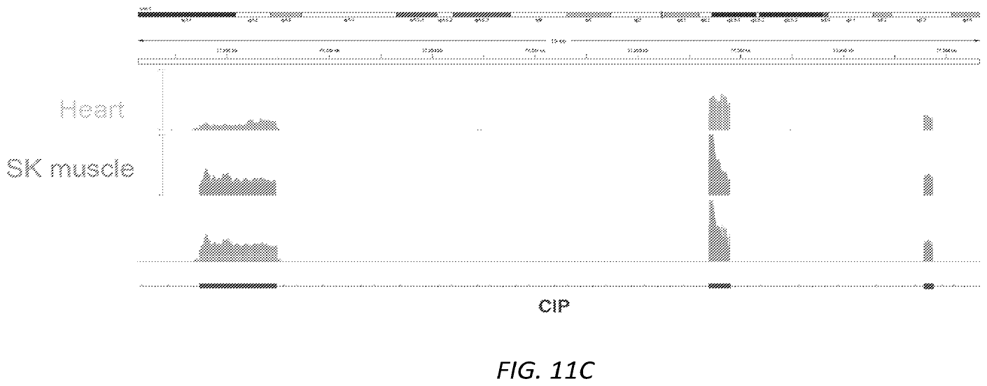

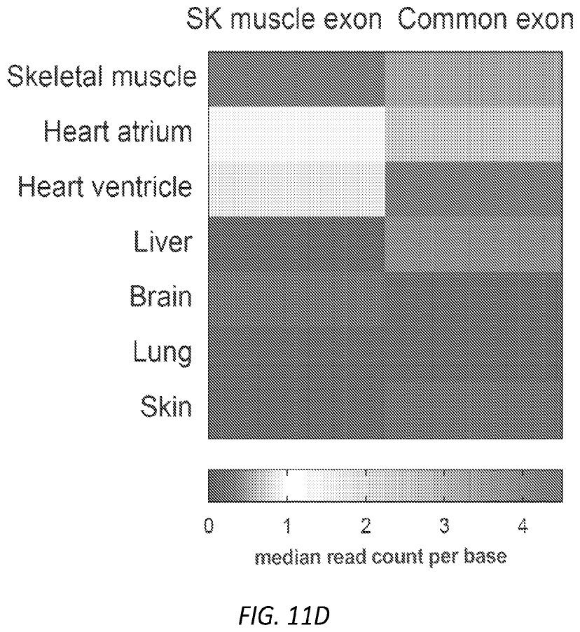

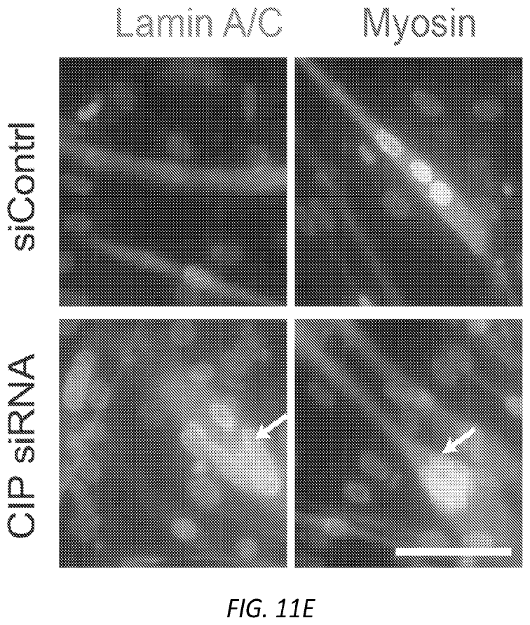

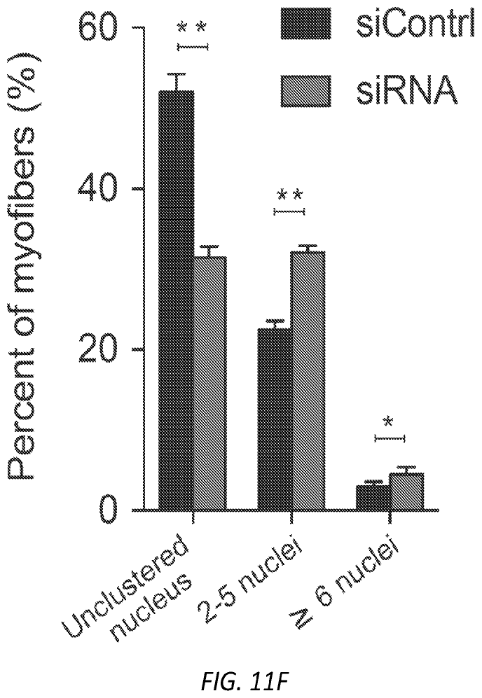

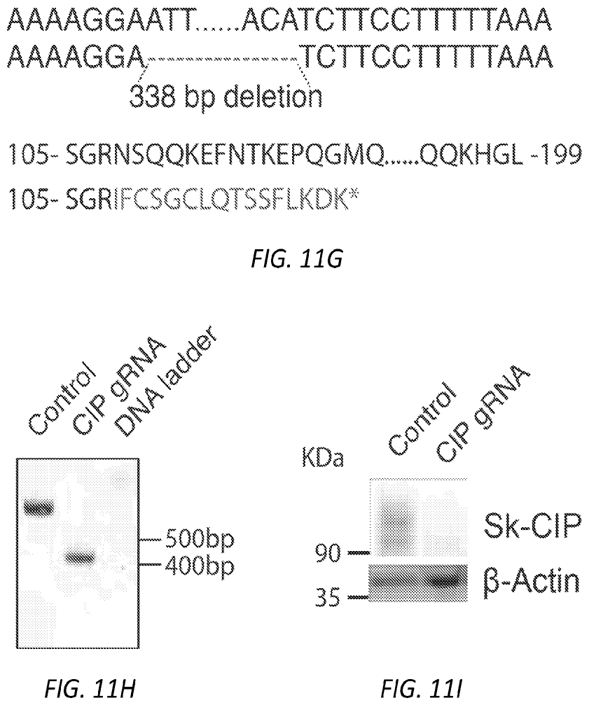

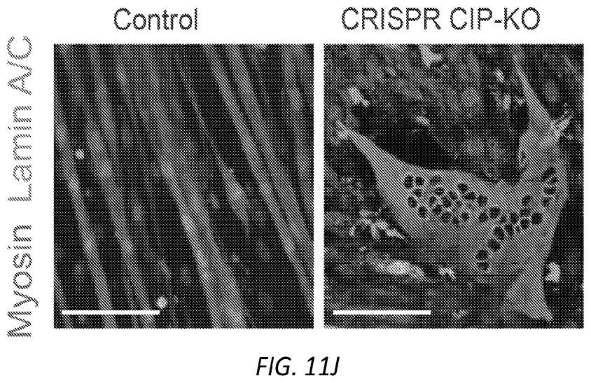

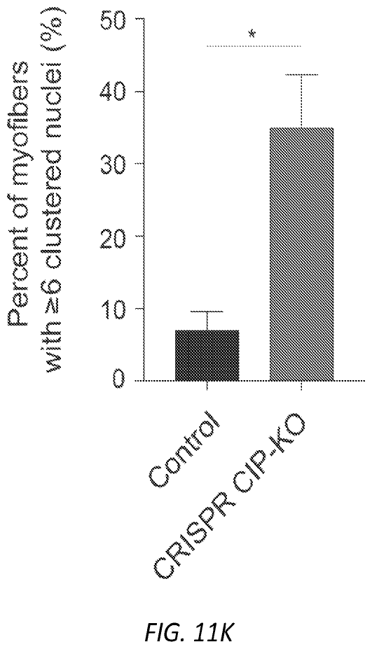

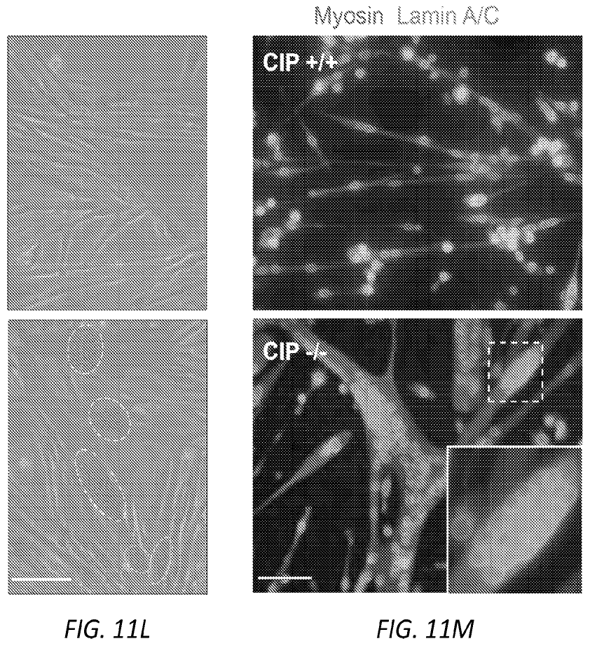

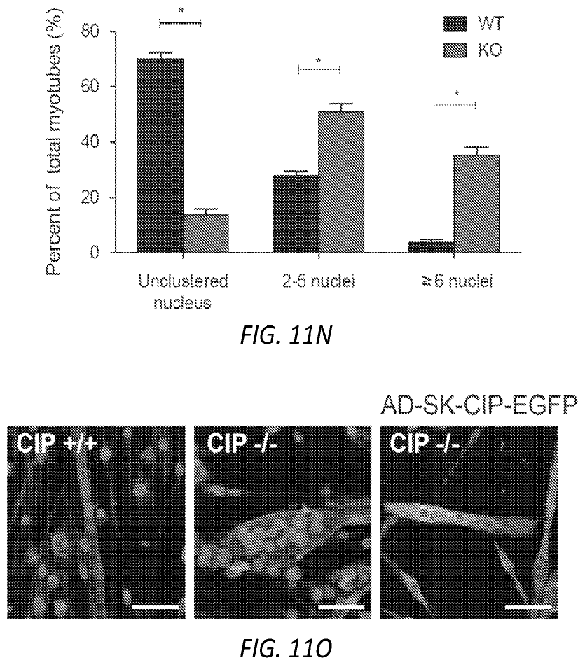

We and others have previously identified the CIP gene (Cardiac Islet-1 interaction Protein), also called Mlip (12, 11), and reported that CIP participates in the regulation of cardiac function in response to stress (14). The CIP gene encodes multiple splicing isoforms, and we discovered an alternatively spliced isoform in skeletal muscle, which we named skeletal muscle CIP (sk-CIP) (FIG. 11A). The CIP sequence is highly conserved among mammals; searching the GTEx Consortium Database reveals that the human homologue of the mouse CIP gene is highly expressed in the heart and skeletal muscle (FIG. 11B). A close examination of the distribution of cardiac and skeletal muscle-specific CIP isoforms from the ENCODE database (FIG. 11C) and the GTEx Consortium Database (FIG. 11D) indicates the existence of cardiac- and skeletal muscle-enriched isoforms in both mouse and human. Interestingly, during C2C12 myoblast differentiation, the expression levels of both sk-CIP and total CIP transcripts are increased, similar to that of myogenin. We detected increased expression of sk-CIP protein during C2C12 differentiation. Furthermore, ChIP-Seq data from ENCODE shows strong binding of MyoD and myogenin to the CIP gene promoter, indicating that sk-CIP muscle specific expression is regulated by myogenic transcription factors during myogenic differentiation.

To begin to assess the function of CIP in skeletal muscle, we first knocked down CIP using a pool of siRNAs in C2C12 cells; interestingly, we found that inhibition of CIP resulted in nuclear clustering within single myotubes without affecting myogenic differentiation gene expression (FIG. 11E, 11F). To further confirm that CIP affects myonuclear positioning, we utilized CRISPR/Cas9 gene editing to create a CIP nonsense mutation to completely abolish CIP expression in C2C12 cells (FIG. 11G, H, I). Loss of CIP expression is made apparent by the loss of multiple bands in western blots in both siRNA knock-down and CRISPR/Cas9 knockout experiments consistent with previous reports of CIP expression (11, 14). CIP knockout C2C12 myoblasts differentiated into myotubes with large numbers of nuclei clustered together within single myotubes (FIG. 11J, 11K), confirming the role of sk-CIP in the regulation of myonuclear positioning. Next, we isolated primary myoblasts from skeletal muscles of CIP knockout mice (11, 14) and subjected them to myogenic differentiation. While wild type myoblasts differentiated into long and thin myofibers, with well separated nuclei and efficient spontaneous contraction (FIG. 11L, M), CIP knockout myoblasts differentiated into myotubes containing large numbers of clustered nuclei (FIG. 11L, M). Quantitative analysis confirms that loss of CIP results in the formation of myonuclear clustering; a significant portion of the CIP mutant myotubes contain more than 6 nuclei clustered together (FIG. 11N). To exclude the possibility that CIP affects myonuclear position indirectly by altering cell proliferation and/or myogenesis, we compared the proliferation and differentiation capacity of WT and CIP-KO myoblasts and found no difference, suggesting that the myonuclear clustering defect is unlikely to be due to myogenic differentiation defects. Finally, we generated an adenovirus to overexpress sk-CIP, and we found that infecting CIP-KO myoblasts with sk-CIP adenovirus could efficiently rescue the myonuclear clustering defect (FIG. 11O), confirming that sk-CIP is responsible for the myonuclear clustering phenotype. Together, these results indicate that CIP is a novel regulator of nuclear positioning in mammalian skeletal muscle.

### CIP is Required for Myonuclear Positioning In Vivo

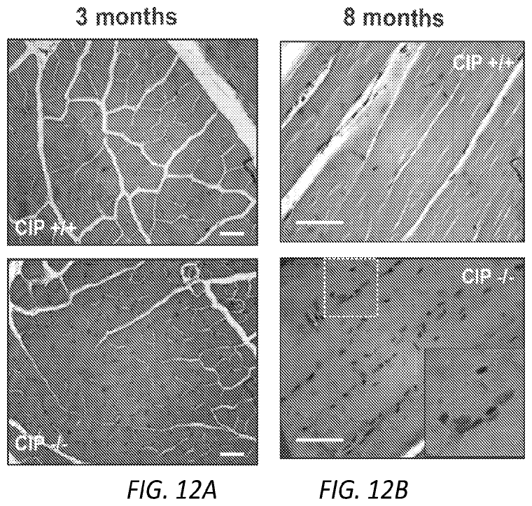

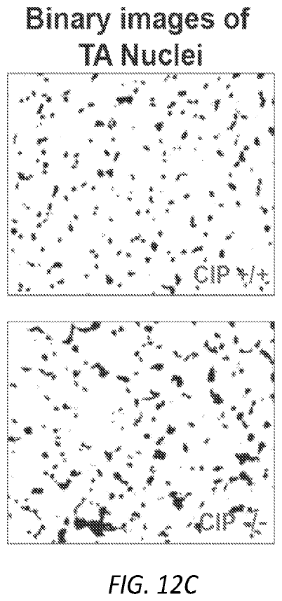

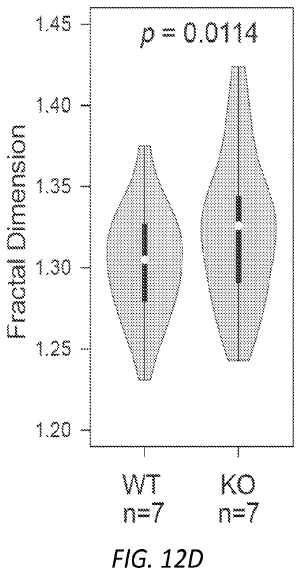

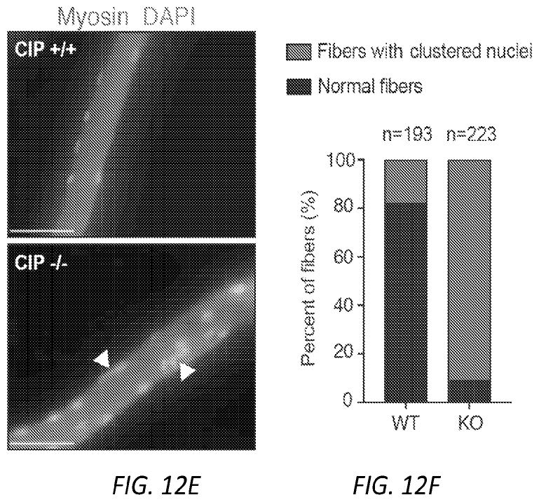

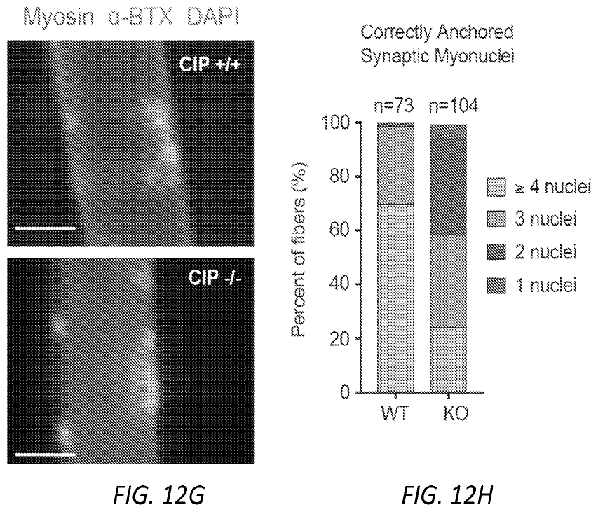

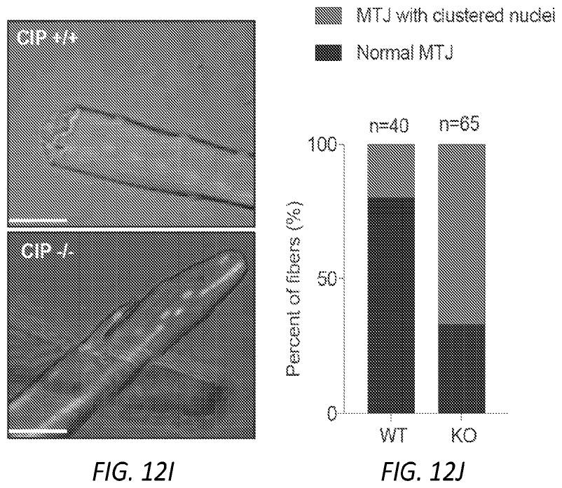

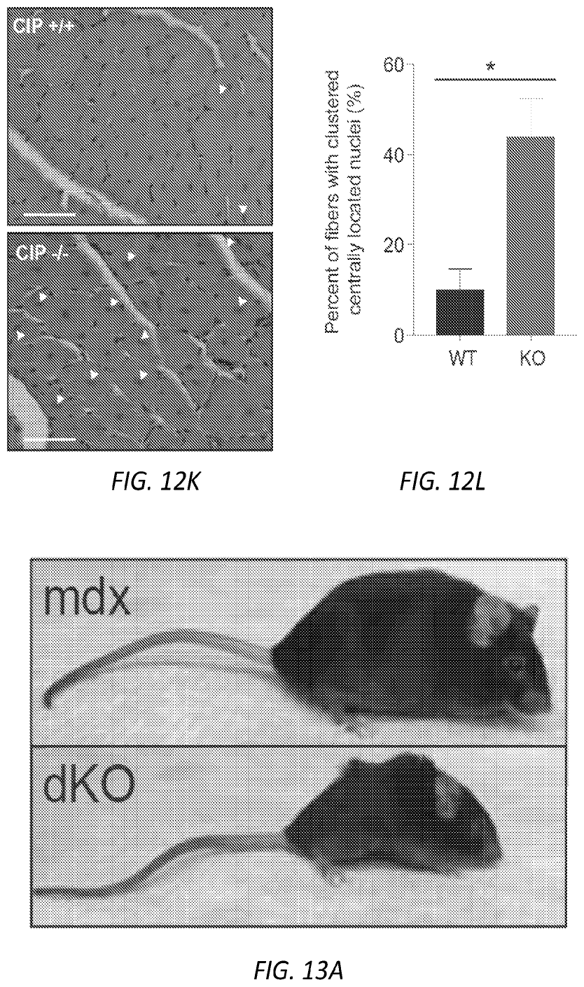

To test if CIP is indeed required for skeletal muscle myonuclear positioning in vivo, we turned to CIP-KO mice (11, 14). The CIP-KO mice were generated in a manner such that both cardiac and skeletal muscle isoforms were abolished (11). CIP-KO mice appear to be normal, without gross morphological defects in skeletal muscle (FIG. 12A). We did not observe changes in fiber type composition nor sarcolemma damage. Skeletal muscle function appears to be normal in young CIP-KO mice. However, histological analysis of skeletal muscle from 8 month old CIP-KO mice reveals severe myonuclear mispositioning in TA muscle (FIG. 12B). The erroneous myonuclear positioning is manifest even more in later life as various large clusters of nuclei inside individual myofibers are readily detected. When fractal dimension analysis was applied to binary images to quantify the pattern of myonuclear localization, CIP-KO muscle shows significantly higher complexity of myonuclear positioning than wild type controls (FIG. 12C, 12D). Analyses and quantification of isolated EDL muscle fibers show that most CIP-KO muscle fibers contain more than three myonuclei clustered together (FIG. 12E, 12F). Analysis of the distance between adjacent nuclei in isolated single TA myofibers shows a significant reduction in the sub-sarcolemma inter-myonuclear distance in CIP-KO muscle. The neuromuscular junction (NMJ) is a specialized structure in muscle fibers that mediates the interaction of motor neuron and muscle fiber (52, 53). In wild type myofibers, NMJs are characterized by acetylcholine receptors (AChR) surrounding clustered myonuclei within the NMJ region; in contrast, myonuclei at the NMJs fail to be engulfed by AChR clustering and tend to be in contact with each other in CIP-KO muscles (FIG. 12G, H). Additionally, at the myotendinous junction (MTJ), where muscle fibers contact tendinous tissues, loss of CIP causes myonuclear disarrangement and clustering, with clusters sometimes centrally located within the MTJ portion of the myofiber (FIG. 121, J). Collectively, these data demonstrate that sk-CIP is required for proper myonuclear positioning in vivo.

Adult skeletal muscle can regenerate in response to damage, owing to the activation of satellite cells, endogenous myogenic stem cells which proliferate, differentiate, and fuse with residual muscle fibers (54, 55). We tested whether CIP is also involved in myonuclear positioning during muscle regeneration. Skeletal muscle from CIP-KO mice regenerates in a similar pattern as that of control mice after cardiotoxin injection-induced degeneration, consistent with the view that CIP does not affect the myogenic differentiation program per se; however, there is a profound clustering of centrally localized nuclei (CLN) in regenerated CIP-KO myofibers (FIG. 12K). Quantitative analysis reveals a greater number of myofibers containing more than 2 clustered CLN in CIP-KO muscle (FIG. 12L). Therefore, we conclude that CIP is required for myonuclear positioning in skeletal muscle fibers during both initial muscle development and adult muscle regeneration.

### Loss of CIP and Dystrophin Results in Severe Muscular Dystrophy

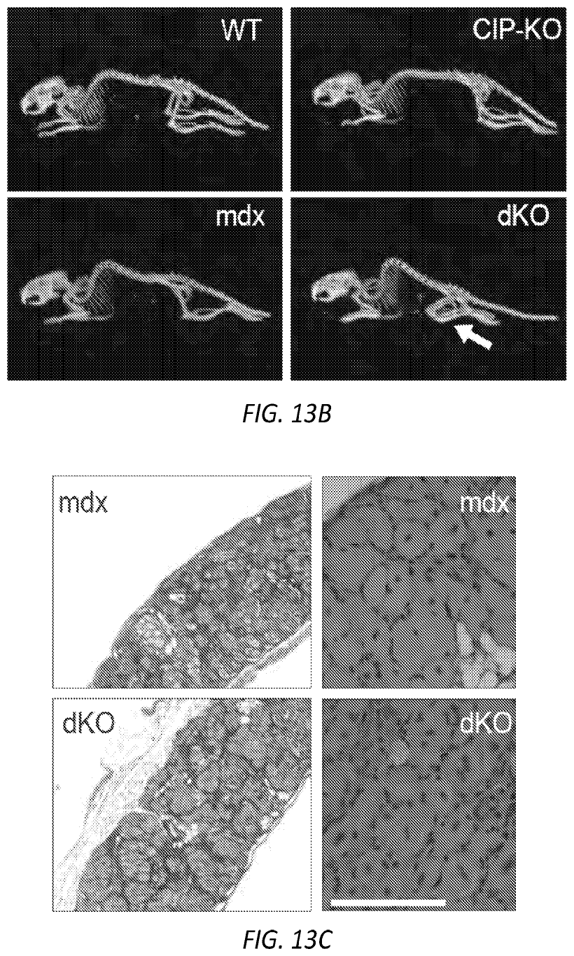

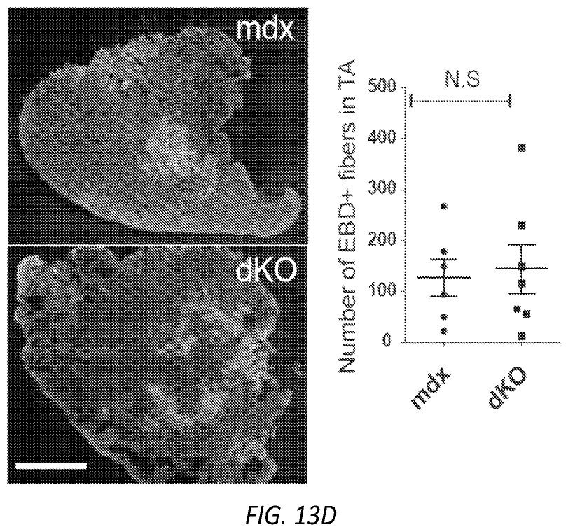

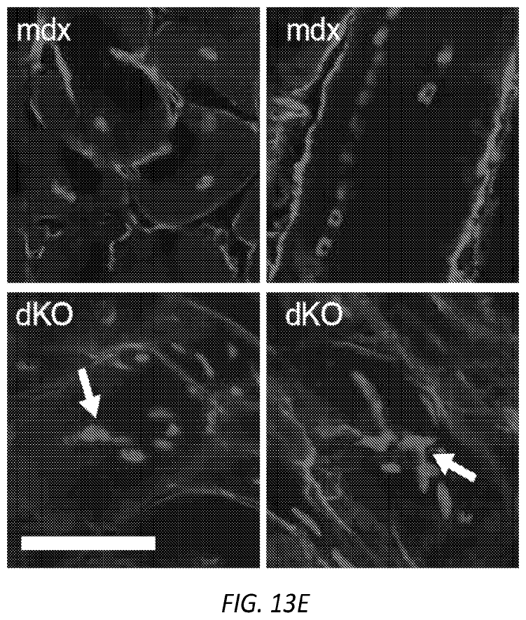

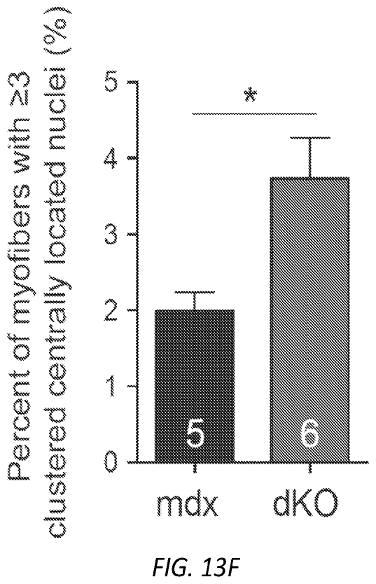

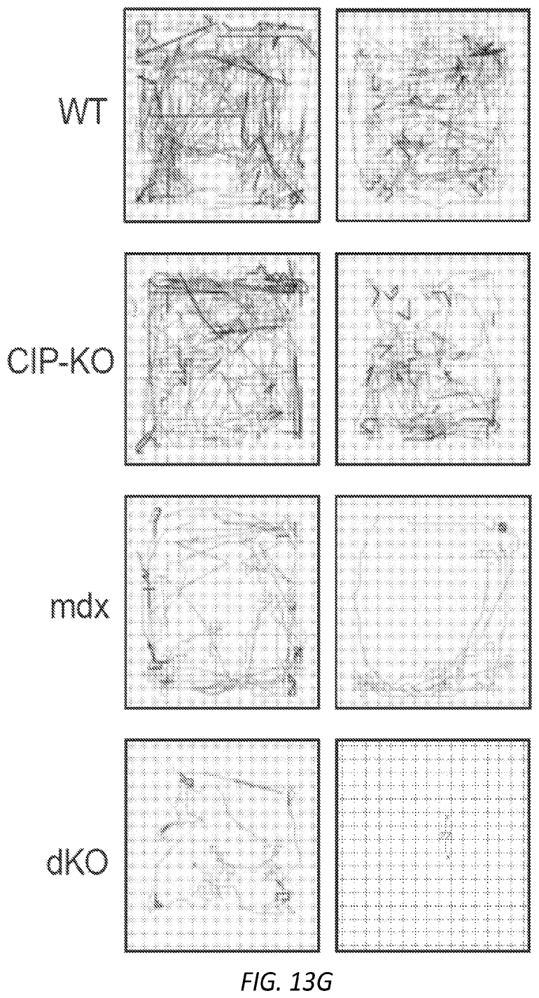

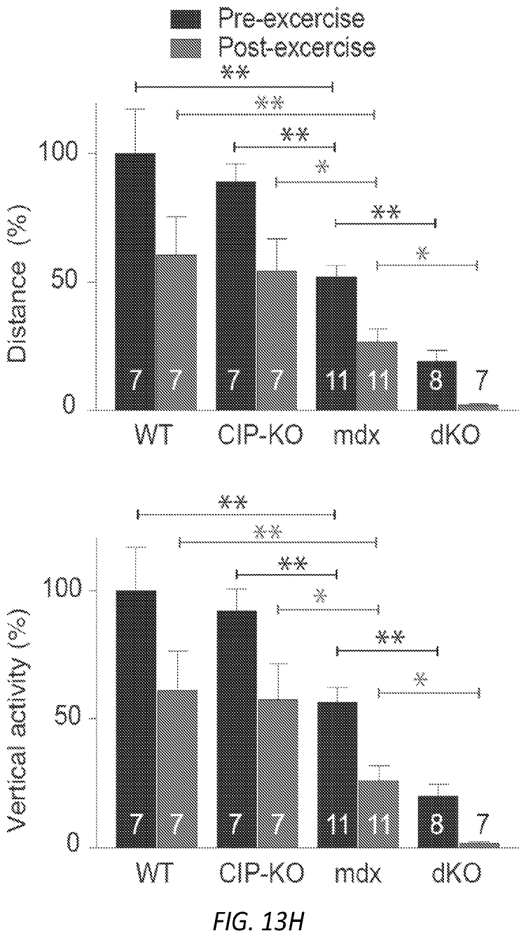

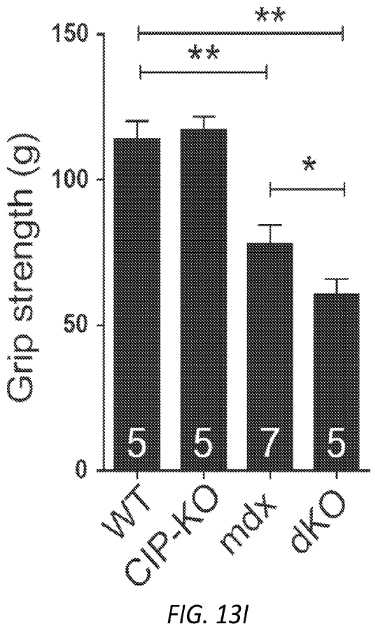

Improperly positioned nuclei are hallmarks of numerous muscle disorders, including Duchenne muscular dystrophy and centronuclear myopathies. Patients suffering from many muscle diseases show a variety degree of muscle weakness with incorrectly localized myonuclei. However, it is unclear whether those incorrectly positioned nuclei contribute to the development of the pathology of such muscle diseases (Meinke et al., PLoS Genet 10, e1004605 (2014)). To investigate the pathological impact of CIP-dependent myonuclear positioning defects in muscle diseases, we bred CIP-KO mice with mdx mice, the most commonly used mouse model of Duchenne muscular dystrophy (Bulfield et al., Proc Natl Acad Sci USA 81, 1189-1192 (1984)). The CIP/mdx double knockout mice (DKO) are smaller than age matched mdx mice and show progressive muscular dystrophy as demonstrated by severe kyphosis (FIG. 13A, B). DKO mice were unable to fully extend their hind limbs during microCT scanning due to skeletal muscle weakness (FIG. 13B, arrow). However, loss of CIP did not increase fibrotic and fatty tissue deposits in skeletal muscles; neither did it increase sarcolemma damage beyond what is found in control mdx muscle (FIG. 13C, D). Nevertheless, we observed significantly more clustered central nuclei in the myofibers of DKO mice (FIG. 13E, F). When subjected to exercise, DKO mice show dramatically lower activity before and after forced exercise (FIG. 13G, H). Most importantly, DKO mice exhibit much weaker skeletal muscle functional output as measured by grip strength (FIG. 131). Together, these data demonstrate that CIP plays an important role in skeletal muscle and muscular dystrophy.

### Example 4. Inhibition of Nox4 Protects Against Dystrophic Cardiomyopathy In Vivo

To examine whether pharmacological inhibition of Nox4 protects against development of cardiomyopathy, we administered a Nox4 Inhibitor to two different mouse models of dystrophic cardiomyopathy.

### Method: Oral Gavage of Nox4 Inhibitor

Nox4 inhibitor GKT 137831 was purchased from MilliporeSigma and dissolved in corn oil.

Male mutation mice for both CIP and dystrophin genes (CIP-KO/mdx dko) were divided into four groups:
1) group young control (3-month old mouse with corn oil);
2) group young inhibitor (3-month old mouse with nox4 inhibitor);
3) group old control (5-month old mouse with corn oil);
4) group Old inhibitor (5-month old mouse with nox4 inhibitor)
   Male mutation mice for utrophin and dystrophin (c) were divided into two groups:
1) group C: control (3-month old mouse with corn oil);
2) group I: inhibitor (3-month old mouse with nox4 inhibitor);
   For all the animals in the inhibitor treatment group, the Nox4 Inhibitor (GKT137831 (SIGMA) diluted 1:100 in corn oil) was applied at the dosage of 60 mg/kg/day daily; control group was treated with corn oil only.

Cardiac function was monitored using echocardiography before (pre-) and after Nox4 Inhibitor treatment at indicated times (weeks or months) and was presented as EF (ejection fraction) or FS % (fractional shortening), respectively. Utrn/mdx dko mice were sacrificed after final echocardiography measurement after 2 months. Histology was performed in the heart tissues and the expression of cardiac genes and fibronectin, a molecular marker for fibrosis, measured using real-time qPCR.

### Results:

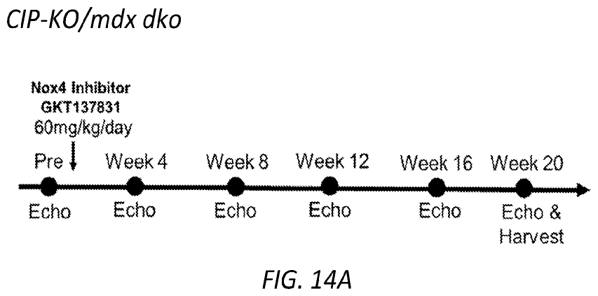

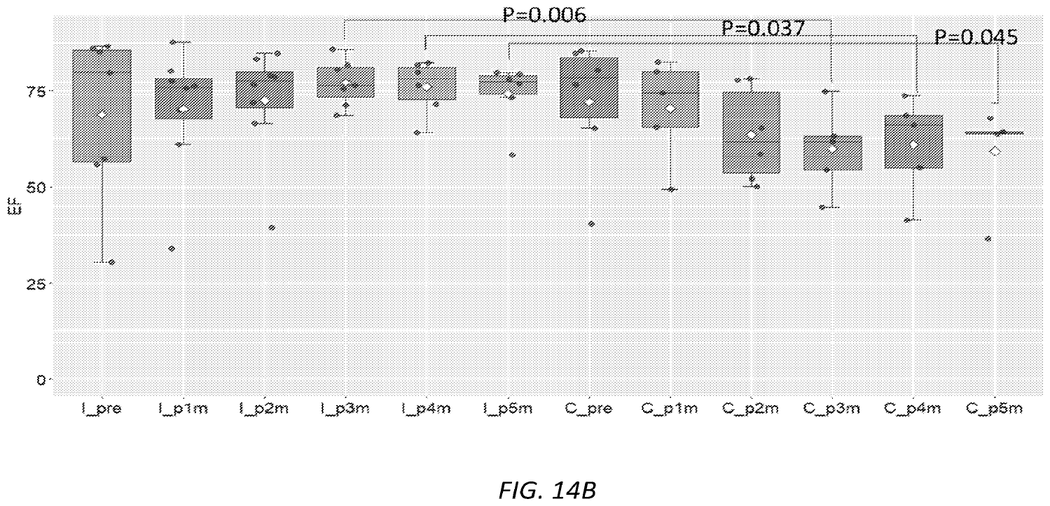

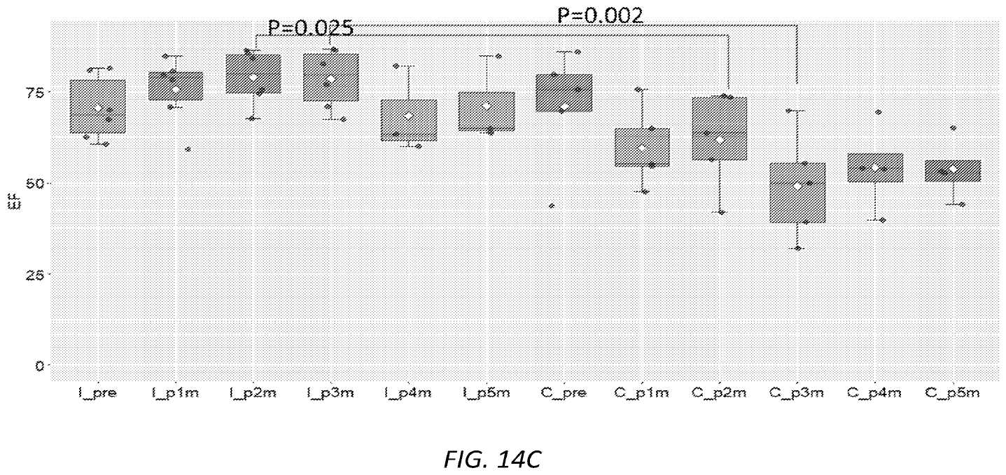

We treated CIP-KO:mdx and control Mdx mice with Nox4 inhibitor (GKT137831, 60 mg/kg/day) daily for 4 weeks (see FIG. 14A). Echocardiography measurement demonstrated showed that the Nox4 inhibitor reduced the left ventricle dilation (LVID) and preserved cardiac function (Ejection Fraction, EF) in both younger (FIG. 14B) and older mice (FIG. 14C).

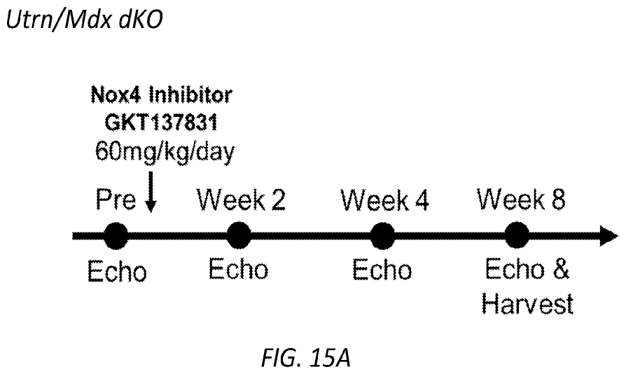

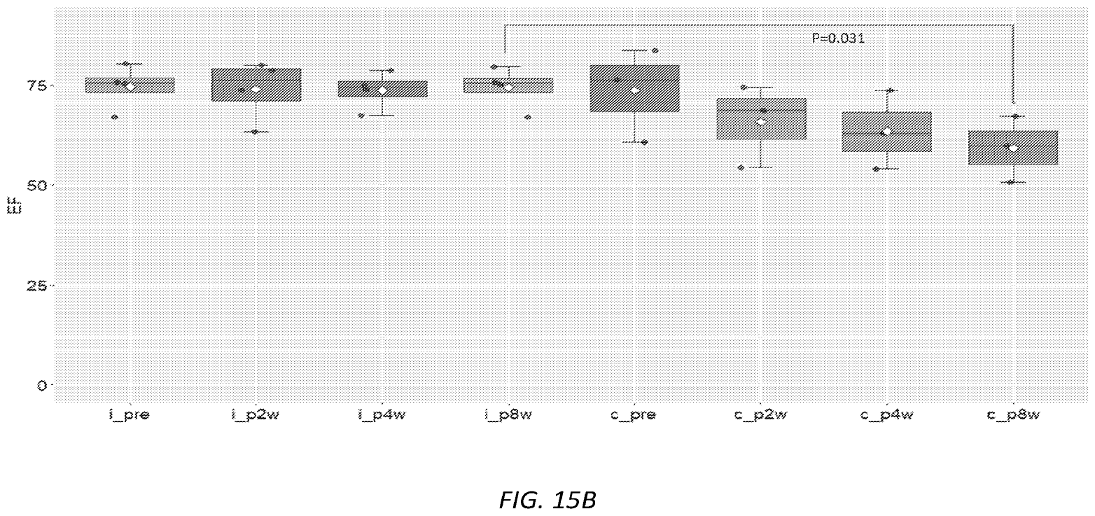

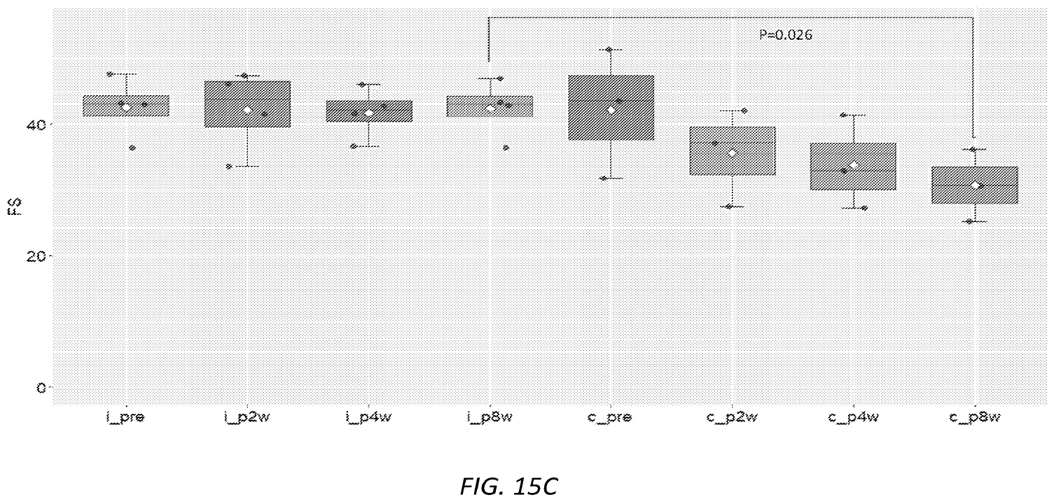

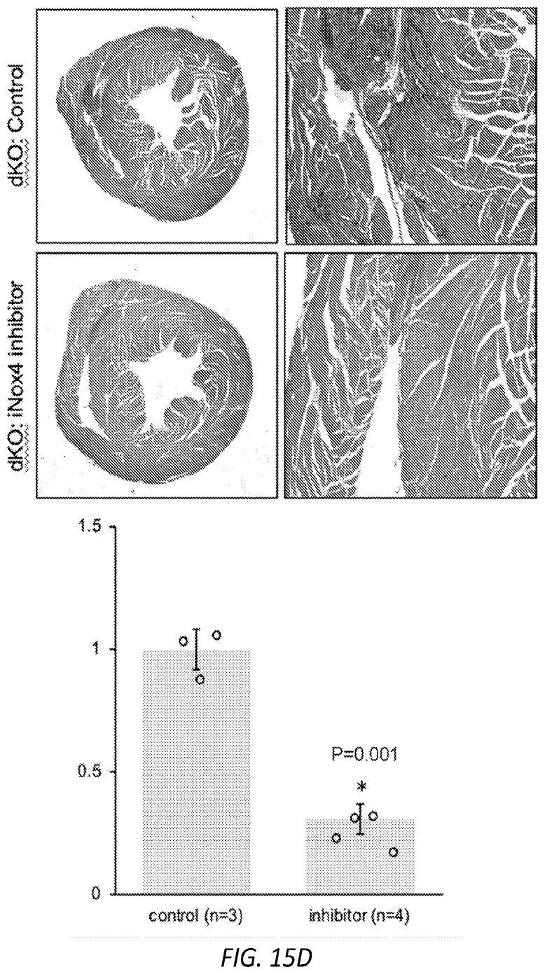

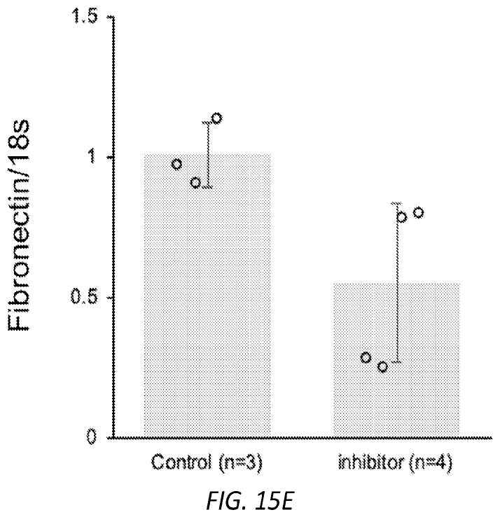

Similar results were seen in Utm/mdx dko mice, treated and evaluated as shown in FIG. 15A. Echocardiography measurement demonstrated showed that the Nox4 inhibitor reduced the left ventricle dilation (LVID) and preserved cardiac function (Ejection Fraction, EF; Fractional Shortening, FS %) in both younger (FIG. 15B) and older mice (FIG. 15C).

Histological analysis confirmed that Nox4 inhibitor treatment reduced the size of the left ventricle dimension; decreased the formation of cardiac fibrosis and the formation of scars (FIG. 15D). Nox4 inhibitor treatment reduced fibronectin expression in the hearts of mdx/Utm^{-/-} (dKO) mice (FIG. 15E). Together, these results show that application of the Nox4 inhibitor GKT137831 to mdx/CIP-KO mice potently ameliorated dystrophic cardiomyopathy, improves cardiac function, and reduces cardiac fibrosis, preventing the development of heart failure.

### REFERENCES

1. Hoffman E P, Brown R H, Jr. and Kunkel L M. Dystrophin: the protein product of the Duchenne muscular dystrophy locus. Cell. 1987; 51:919-28.
2. Monaco A P, Neve R L, Colletti-Feener C, Bertelson C J, Kurnit D M and Kunkel L M. Isolation of candidate cDNAs for portions of the Duchenne muscular dystrophy gene. Nature. 1986; 323:646-50.
3. Chamberlain J R and Chamberlain J S. Progress toward Gene Therapy for Duchenne Muscular Dystrophy. Molecular therapy: the journal of the American Society of Gene Therapy. 2017; 25:1125-1131.
4. Guiraud S and Davies K E. Pharmacological advances for treatment in Duchenne muscular dystrophy. Curr Opin Pharmacol. 2017; 34:36-48.
5. Long C, Amoasii L, Mireault A A, McAnally J R, Li H, Sanchez-Ortiz E, Bhattacharyya S, Shelton J M, Bassel-Duby R and Olson E N. Postnatal genome editing partially restores dystrophin expression in a mouse model of muscular dystrophy. Science. 2016; 351:400-3.
6. Long C, McAnally J R, Shelton J M, Mireault A A, Bassel-Duby R and Olson E N. Prevention of muscular dystrophy in mice by CRISPR/Cas9-mediated editing of germline DNA. Science. 2014; 345:1184-1188.
7. Min Y L, Bassel-Duby R and Olson E N. CRISPR Correction of Duchenne Muscular Dystrophy. Annu Rev Med. 2019; 70:239-255.
8. Amoasii L, Hildyard J C W, Li H, Sanchez-Ortiz E, Mireault A, Caballero D, Harron R, Stathopoulou T R, Massey C, Shelton J M, Bassel-Duby R, Piercy R J and Olson E N. Gene editing restores dystrophin expression in a canine model of Duchenne muscular dystrophy. Science. 2018; 362:86-91.
9. Kamdar F and Garry D J. Dystrophin-Deficient Cardiomyopathy. J Am Coll Cardiol. 2016; 67:2533-46.
10. McNally E M. New approaches in the therapy of cardiomyopathy in muscular dystrophy. Annu Rev Med. 2007; 58:75-88.
11. Huang Z P, Young Seok H, Zhou B, Chen J, Chen J F, Tao Y, Pu W T and Wang D Z. CIP, a cardiac Isl1-interacting protein, represses cardiomyocyte hypertrophy. Circ Res. 2012; 110:818-30.
12. Ahmady E, Deeke S A, Rabaa S, Kouri L, Kenney L, Stewart A F and Burgon P G. Identification of a novel muscle A-type lamin-interacting protein (MLIP). J Biol Chem. 2011; 286:19702-13.
13. Cattin M E, Wang J, Weldrick J J, Roeske C L, Mak E, Thom S L, DaSilva J N, Wang Y, Lusis A J and Burgon P G. Deletion of MLIP (muscle-enriched A-type lamin-interacting protein) leads to cardiac hyperactivation of Akt/mammalian target of rapamycin (mTOR) and impaired cardiac adaptation. J Biol Chem. 2015; 290:26699-714.
14. Huang Z P, Kataoka M, Chen J, Wu G, Ding J, Nie M, Lin Z, Liu J, Hu X, Ma L, Zhou B, Wakimoto H, Zeng C, Kyselovic J, Deng Z L, Seidman C E, Seidman J G, Pu W T and Wang D Z. Cardiomyocyte-enriched protein CIP protects against pathophysiological stresses and regulates cardiac homeostasis. J Clin Invest. 2015; 125:4122-34.
15. Bulfield G, Siller W G, Wight P A and Moore K J. X chromosome-linked muscular dystrophy (mdx) in the mouse. Proceedings of the National Academy of Sciences of the United States of America. 1984; 81:1189-92.
16. Bruning J C, Michael M D, Winnay J N, Hayashi T, Horsch D, Accili D, Goodyear L J and Kahn C R. A muscle-specific insulin receptor knockout exhibits features of the metabolic syndrome of NIDDM without altering glucose tolerance. Mol Cell. 1998; 2:559-69.
17. Molkentin J D, Lu J R, Antos C L, Markham B, Richardson J, Robbins J, Grant S R and Olson E N. A calcineurin-dependent transcriptional pathway for cardiac hypertrophy. Cell. 1998; 93:215-28.
18. Grieger J C, Choi V W and Samulski R J. Production and characterization of adeno-associated viral vectors. Nature protocols. 2006; 1:1412-28.
19. O'Connell T D, Swigart P M, Rodrigo M C, Ishizaka S, Joho S, Tumbull L, Tecott L H, Baker A J, Foster E, Grossman W and Simpson P C. Alphal-adrenergic receptors prevent a maladaptive cardiac response to pressure overload. J Clin Invest. 2006; 116:1005-15.
20. Xing W, Zhang T C, Cao D, Wang Z, Antos C L, Li S, Wang Y, Olson E N and Wang D Z. Myocardin induces cardiomyocyte hypertrophy. Circ Res. 2006; 98:1089-97.
21. Bridges L R. The association of cardiac muscle necrosis and inflammation with the degenerative and persistent myopathy of MDX mice. J Neurol Sci. 1986; 72:147-57.
22. Quinlan J G, Hahn H S, Wong B L, Lorenz J N, Wenisch A S and Levin L S. Evolution of the mdx mouse cardiomyopathy: physiological and morphological findings. Neuromuscul Disord. 2004; 14:491-6.
23. Walcher T, Steinbach P, Spiess J, Kunze M, Gradinger R, Walcher D and Bernhardt P. Detection of long-term progression of myocardial fibrosis in Duchenne muscular dystrophy in an affected family: a cardiovascular magnetic resonance study. Eur J Radiol. 2011; 80:115-9.
24. Kuroda J, Ago T, Matsushima S, Zhai P, Schneider M D and Sadoshima J. NADPH oxidase 4 (Nox4) is a major source of oxidative stress in the failing heart. Proc Natl Acad Sci USA. 2010; 107:15565-70.
25. Matsushima S, Kuroda J, Ago T, Zhai P, Park J Y, Xie L H, Tian B and Sadoshima J. Increased oxidative stress in the nucleus caused by Nox4 mediates oxidation of HDAC4 and cardiac hypertrophy. Circ Res. 2013; 112:651-63.
26. Matsushima S, Kuroda J, Zhai P, Liu T, Ikeda S, Nagaraj an N, Oka S, Yokota T, Kinugawa S, Hsu C P, Li H, Tsutsui H and Sadoshima J. Tyrosine kinase FYN negatively regulates NOX4 in cardiac remodeling. J Clin Invest. 2016; 126:3403-16.
27. Owen J B and Butterfield D A. Measurement of oxidized/reduced glutathione ratio. Methods Mol Biol. 2010; 648:269-77.
28. Wilkins B J, Dai Y S, Bueno O F, Parsons S A, Xu J, Plank D M, Jones F, Kimball T R and Molkentin J D. Calcineurin/NFAT coupling participates in pathological, but not physiological, cardiac hypertrophy. Circ Res. 2004; 94:110-8.
29. Ni Y G, Berenji K, Wang N, Oh M, Sachan N, Dey A, Cheng J, Lu G, Morris D J, Castrillon D H, Gerard R D, Rothermel B A and Hill J A. Foxo transcription factors blunt cardiac hypertrophy by inhibiting calcineurin signaling. Circulation. 2006; 114:1159-68.
30. Berry J M, Le V, Rotter D, Battiprolu P K, Grinsfelder B, Tannous P, Burchfield J S, Czubryt M, Backs J, Olson E N, Rothermel B A and Hill J A. Reversibility of adverse, calcineurin-dependent cardiac remodeling. Circ Res. 2011; 109:407-17.
31. Williams C R and Gooch J L. Calcineurin Abeta regulates NADPH oxidase (Nox) expression and activity via nuclear factor of activated T cells (NFAT) in response to high glucose. J Biol Chem. 2014; 289:4896-905.
32. Heineke J, Auger-Messier M, Correll R N, Xu J, Benard M J, Yuan W, Drexler H, Parise L V and Molkentin J D. CIB1 is a regulator of pathological cardiac hypertrophy. Nat Med. 2010; 16:872-9.
33. Taigen T, De Windt L J, Lim H W and Molkentin J D. Targeted inhibition of calcineurin prevents agonist-induced cardiomyocyte hypertrophy. Proc Natl Acad Sci USA. 2000; 97:1196-201.
34. Heineke J and Molkentin J D. Regulation of cardiac hypertrophy by intracellular signalling pathways. Nat Rev Mol Cell Biol. 2006; 7:589-600.
35. Serrano A L and Munoz-Canoves P. Fibrosis development in early-onset muscular dystrophies: Mechanisms and translational implications. Semin Cell Dev Biol. 2017; 64:181-190.
36. Moulin M and Ferreiro A. Muscle redox disturbances and oxidative stress as pathomechanisms and therapeutic targets in early-onset myopathies. Semin Cell Dev Biol. 2017; 64:213-223.
37. Allen D G, Whitehead N P and Froehner S C. Absence of Dystrophin Disrupts Skeletal Muscle Signaling: Roles of Ca2+, Reactive Oxygen Species, and Nitric Oxide in the Development of Muscular Dystrophy. Physiol Rev. 2016; 96:253-305.
38. Dombernowsky N W, Olmestig J N E, Witting N and Kruuse C. Role of neuronal nitric oxide synthase (nNOS) in Duchenne and Becker muscular dystrophies—Still a possible treatment modality? Neuromuscul Disord. 2018; 28:914-926.
39. Russo V, Papa A A, Williams E A, Rago A, Palladino A, Politano L and Nigro G. ACE inhibition to slow progression of myocardial fibrosis in muscular dystrophies. Trends Cardiovasc Med. 2018; 28:330-337.
40. Goldfine A B and Shoelson S E. Therapeutic approaches targeting inflammation for diabetes and associated cardiovascular risk. J Clin Invest. 2017; 127:83-93.
41. Spumey C F. Cardiomyopathy of Duchenne muscular dystrophy: current understanding and future directions. Muscle & nerve. 2011; 44:8-19.
42. Whitehead N P, Yeung E W and Allen D G. Muscle damage in mdx (dystrophic) mice: role of calcium and reactive oxygen species. Clin Exp Pharmacol Physiol. 2006; 33:657-62.
43. Betts C A, McClorey G, Healicon R, Hammond S M, Manzano R, Muses S, Ball V, Godfrey C, Merritt T M, van Westering T, O'Donovan L, Wells K E, Gait M J, Wells D J, Tyler D and Wood M J. Cmah-dystrophin deficient mdx mice display an accelerated cardiac phenotype that is improved following peptide-PMO exon skipping treatment. Hum Mol Genet. 2019; 28:396-406.
44. Spumey C F, Knoblach S, Pistilli E E, Nagaraju K, Martin G R and Hoffman E P. Dystrophin-deficient cardiomyopathy in mouse: expression of Nox4 and Lox are associated with fibrosis and altered functional parameters in the heart. Neuromuscul Disord. 2008; 18:371-81.
45. Kyrychenko V, Polakova E, Janicek R and Shirokova N. Mitochondrial dysfunctions during progression of dystrophic cardiomyopathy. Cell Calcium. 2015; 58:186-95.
46. Khouzami L, Bourin M C, Christov C, Damy T, Escoubet B, Caramelle P, Perier M, Wahbi K, Meune C, Pavoine C and Pecker F. Delayed cardiomyopathy in dystrophin deficient mdx mice relies on intrinsic glutathione resource. Am J Pathol. 2010; 177:1356-64.
47. Kyrychenko S, Kyrychenko V, Badr M A, Ikeda Y, Sadoshima J and Shirokova N. Pivotal role of miR-448 in the development of ROS-induced cardiomyopathy. Cardiovasc Res. 2015; 108:324-34.
48. Gonzalez D R, Treuer A V, Lamirault G, Mayo V, Cao Y, Dulce R A and Hare J M. NADPH oxidase-2 inhibition restores contractility and intracellular calcium handling and reduces arrhythmogenicity in dystrophic cardiomyopathy. Am J Physiol Heart Circ Physiol. 2014; 307:H710-21.
49. Ago T, Kuroda J, Pain J, Fu C, Li H and Sadoshima J. Upregulation of Nox4 by hypertrophic stimuli promotes apoptosis and mitochondrial dysfunction in cardiac myocytes. Circ Res. 2010; 106:1253-64.
50. Esslinger U, Gamier S, Komiat A, Proust C, Kararigas G, Muller-Nurasyid M, Empana J P, Morley M P, Perret C, Stark K, Bick A G, Prasad S K, Kriebel J, Li J, Tiret L, Strauch K, O'Regan D P, Marguiles K B, Seidman J G, Boutouyrie P, Lacolley P, Jouven X, Hengstenberg C, Komajda M, Hakonarson H, Isnard R, Arbustini E, Grallert H, Cook S A, Seidman C E, Regitz-Zagrosek V, Cappola T P, Charron P, Cambien F and Villard E. Exome-wide association study reveals novel susceptibility genes to sporadic dilated cardiomyopathy. PLoS One. 2017; 12:e0172995.
51. He A, Kong S W, Ma Q, Pu W T. Co-occupancy by multiple cardiac transcription factors identifies transcriptional enhancers active in heart. Proceedings of the National Academy of Sciences of the United States of America. 2011; 108:5632-5637
52. R. Rudolf, M. R. Deschenes, M. Sandri, Neuromuscular junction degeneration in muscle wasting. Curr Opin Clin Nutr Metab Care 19, 177-181 (2016).
53. L. Li, W. C. Xiong, L. Mei, Neuromuscular Junction Formation, Aging, and Disorders. Annu Rev Physiol 80, 159-188 (2018).
54. S. D. Gopinath, T. A. Rando, Stem cell review series: aging of the skeletal muscle stem cell niche. Aging Cell 7, 590-598 (2008).
55. S. Kuang, M. A. Rudnicki, The emerging biology of satellite cells and their therapeutic potential. Trends Mol Med 14, 82-91 (2008).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating cardiomyopathy or heart failure in a subject, the method comprising administering to a subject in need thereof a therapeutically effective amount of an inhibitor of NADPH oxidase 4 (Nox4).

2. The method of claim 1, wherein the inhibitor is administered daily.

3. The method of claim 1, wherein the inhibitor is selected from the group consisting of GKT137831; GKT136901; GSK2795039; VAS2870; perhexiline; VAS3947; compound 87 (2-(2-chlorophenyl)-5-[(1-methylpyrazol-3-yl)methyl]-4-[[methyl(pyridin-3-ylmethyl)amino]methyl]-1H-pyrazolo[4,3-c]pyridine-3,6-dione); compound 7c (10-benzyl-2-(2-chlorophenyl)-7,8,9,11-tetrahydro-3H-pyrazolo[4,5]pyrido[5,6-a][1,4]diazepine-1,5-dione; APX-115; VAS2870; fulvene-5; grindelic acid; phenantridinones; fluvenazine; DPI; suramin; ebselen; perhexiline; perhenazine; fluphenazine; and tertiary sulfonylureas.

4. The method of claim 1, wherein the subject has a muscular dystrophy.

5. The method of claim 4, wherein the subject has Duchenne muscular dystrophy (DMD), Becker muscular dystrophy (BMD), and X-linked dilated cardiomyopathy (XL-DCM).

6. The method of claim 1, wherein the subject has sinus tachycardia, atrial arrhythmias, including atrial fibrillation, atrial flutter, atrial tachycardias, and/or left ventricular dysfunction.

7. A method of reducing risk of development of cardiomyopathy or heart failure in a subject, the method comprising administering to a subject in need thereof a therapeutically effective amount of an inhibitor of NADPH oxidase 4 (Nox4).

8. The method of claim 7, wherein the inhibitor is administered daily.

9. The method of claim 7, wherein the inhibitor is selected from the group consisting of GKT137831; GKT136901; GSK2795039; VAS2870; perhexiline; VAS3947; compound 87 (2-(2-chlorophenyl)-5-[(1-methylpyrazol-3-yl)methyl]-4-[[methyl(pyridin-3-ylmethyl)amino]methyl]-1H-pyrazolo[4,3-c]pyridine-3,6-dione); compound 7c (10-benzyl-2-(2-chlorophenyl)-7,8,9,11-tetrahydro-3H-pyrazolo[4,5]pyrido[5,6-a][1,4]diazepine-1,5-dione; APX-115; VAS2870; fulvene-5; grindelic acid; phenantridinones; fluvenazine; DPI; suramin; ebselen; perhexiline; perhenazine; fluphenazine; and tertiary sulfonylureas.

10. The method of claim 7, wherein the subject has a muscular dystrophy.

11. The method of claim 10, wherein the subject has Duchenne muscular dystrophy (DMD), Becker muscular dystrophy (BMD), and X-linked dilated cardiomyopathy (XL-DCM).

12. The method of claim 11, wherein the subject has DMD and is less than 10-12 years of age.

13. The method of claim 7, wherein the subject has left ventricular (LV) strain defects or myocardial fibrosis, but does not have left ventricular dysfuction.

14.-26. (canceled)

27. A method of treating a subject who has a muscular dystrophy, the method comprising administering to the subject a therapeutically effective amount of an inhibitor of NADPH oxidase 4 (Nox4).

28. The method of claim 27, wherein the inhibitor is administered daily.

29. The method of claim 27, wherein the inhibitor is selected from the group consisting of GKT137831; GKT136901; GSK2795039; VAS2870; perhexiline; VAS3947; compound 87 (2-(2-chlorophenyl)-5-[(1-methylpyrazol-3-yl)methyl]-4-[[methyl(pyridin-3-ylmethyl)amino]methyl]-1H-pyrazolo[4,3-c]pyridine-3,6-dione); compound 7c (10-benzyl-2-(2-chlorophenyl)-7,8,9,11-tetrahydro-3H-pyrazolo[4,5]pyrido[5,6-a][1,4]diazepine-1,5-dione; APX-115; VAS2870; fulvene-5; grindelic acid; phenantridinones; fluvenazine; DPI; suramin; ebselen; perhexiline; perhenazine; fluphenazine; and tertiary sulfonylureas.

30. The method of claim 27, wherein the subject has Duchenne muscular dystrophy (DMD), Becker muscular dystrophy (BMD), and X-linked dilated cardiomyopathy (XL-DCM).

31. The method of claim 30, wherein the subject has DMD and is less than 10-12 years of age.

32. The method of claim 6, wherein the left ventricular dysfunction comprises left ventricular ejection fraction (LVEF)<35%.


SEQUENCE LISTING (68 sequences)

SEQ ID NO: 1 - DNA, 23 bp, Artificial
  primer for NPPA
cacagatctgatggatttcaaga

SEQ ID NO: 2 - DNA, 20 bp, Artificial
  primer for NPPA
cctcatcttctaccggcatc

SEQ ID NO: 3 - DNA, 21 bp, Artificial
  primer for NPPB
gtcagtcgtttgggctgtaac

SEQ ID NO: 4 - DNA, 20 bp, Artificial
  primer for NPPB
agacccaggcagagtcagaa

SEQ ID NO: 5 - DNA, 19 bp, Artificial
  primer for FBN1
ccttcctgtggctccagat

SEQ ID NO: 6 - DNA, 18 bp, Artificial
  primer for FBN1
gctgcccccattcataca

SEQ ID NO: 7 - DNA, 20 bp, Artificial
  primer for Acta1
gcccatctatgagggctatg

SEQ ID NO: 8 - DNA, 20 bp, Artificial
  primer for Acta1
aatctcacgttcagctgtgg

SEQ ID NO: 9 - DNA, 20 bp, Artificial
  primer for Tgfb1
aagacttcaccccaaagctg

SEQ ID NO: 10 - DNA, 19 bp, Artificial
  primer for Tgfb1
gagagagggtcctgggatg

SEQ ID NO: 11 - DNA, 20 bp, Artificial
  primer for Des
gcgtgacaacctgatagacg

SEQ ID NO: 12 - DNA, 22 bp, Artificial
  primer for Des
tggatttcctcctgtagtttgg

SEQ ID NO: 13 - DNA, 18 bp, Artificial
  primer for Bax
gtgagcggctgcttgtct

SEQ ID NO: 14 - DNA, 18 bp, Artificial
  primer for Bax
gtgggggtcccgaagtag

SEQ ID NO: 15 - DNA, 20 bp, Artificial
  primer for Cebpa
cgctggtgatcaaacaagag

SEQ ID NO: 16 - DNA, 18 bp, Artificial
  primer for Cebpa
ggtggctggtaggggaag

SEQ ID NO: 17 - DNA, 18 bp, Artificial
  primer for Cebpb
tgatgcaatccggatcaa

SEQ ID NO: 18 - DNA, 19 bp, Artificial
  primer for Cebpb
cacgtgtgttgcgtcagtc

SEQ ID NO: 19 - DNA, 18 bp, Artificial
  primer for Cox6a2
gagcgcccagagttcatc

SEQ ID NO: 20 - DNA, 19 bp, Artificial
  primer for Cox6a2
tgtggaaaagcgtgtggtt

SEQ ID NO: 21 - DNA, 19 bp, Artificial
  primer for Cox8b
agccaaaactcccacttcc

SEQ ID NO: 22 - DNA, 19 bp, Artificial
  primer for Cox8b
gaaccatgaagccaacgac

SEQ ID NO: 23 - DNA, 21 bp, Artificial
  primer for Rictor
ggtgataactacgttcgtcgc

SEQ ID NO: 24 - DNA, 20 bp, Artificial
  primer for Rictor
aaaggtgtacgggcaggtag

SEQ ID NO: 25 - DNA, 19 bp, Artificial
  primer for Bbc3
ttctccggagtgttcatgc

SEQ ID NO: 26 - DNA, 18 bp, Artificial
  primer for Bbc3
tacagcggagggcatcag

SEQ ID NO: 27 - DNA, 19 bp, Artificial
  primer for Ndufa1
tgatggaacgcgatagacg

SEQ ID NO: 28 - DNA, 20 bp, Artificial
  primer for Ndufa1
gccaggaaaatgcttcctta

SEQ ID NO: 29 - DNA, 21 bp, Artificial
  primer for Ndufb9
tttccaagagagagcagtgga

SEQ ID NO: 30 - DNA, 20 bp, Artificial
  primer for Ndufb9
ctcctgcagctgcttaacct

SEQ ID NO: 31 - DNA, 20 bp, Artificial
  primer for Uqcr10
acttccacctttgccctcac

SEQ ID NO: 32 - DNA, 20 bp, Artificial
  primer for Uqcr10
tccacagtttcccctcgttg

SEQ ID NO: 33 - DNA, 18 bp, Artificial
  primer for Uqcr11
ccacaggcctcgatggta

SEQ ID NO: 34 - DNA, 20 bp, Artificial
  primer for Uqcr11
gcagccctagtgtctgtcaa

SEQ ID NO: 35 - DNA, 23 bp, Artificial
  primer for Cox5a
ttaaatgaattgggaatctccac

SEQ ID NO: 36 - DNA, 19 bp, Artificial
  primer for Cox5a
gtccttaggaagcccatcg

SEQ ID NO: 37 - DNA, 19 bp, Artificial
  primer for Sod3
ctcttgggagagcctgaca

SEQ ID NO: 38 - DNA, 20 bp, Artificial
  primer for Sod3
gccagtagcaagccgtagaa

SEQ ID NO: 39 - DNA, 20 bp, Artificial
  primer for Junb
ccacggagggagagaaaatc

SEQ ID NO: 40 - DNA, 19 bp, Artificial
  primer for Junb
agttggcagctgtgcgtaa

SEQ ID NO: 41 - DNA, 21 bp, Artificial
  primer for Fgfr1
gacctacgttcaagcagttgg

SEQ ID NO: 42 - DNA, 18 bp, Artificial
  primer for Fgfr1
tccagcggtatggacagg

SEQ ID NO: 43 - DNA, 19 bp, Artificial
  primer for Smad7
acccccatcaccttagtcg

SEQ ID NO: 44 - DNA, 22 bp, Artificial
  primer for Smad7
gaaaatccattgggtatctgga

SEQ ID NO: 45 - DNA, 20 bp, Artificial
  primer for Rhoc
aaggacctgaggcaagatga

SEQ ID NO: 46 - DNA, 20 bp, Artificial
  primer for Rhoc
aaggcactgatcctgtttgc

SEQ ID NO: 47 - DNA, 18 bp, Artificial
  primer for Pdgfb
cgagggaggaggagccta

SEQ ID NO: 48 - DNA, 19 bp, Artificial
  primer for Pdgfb
gtcttgcactcggcgatta

SEQ ID NO: 49 - DNA, 20 bp, Artificial
  primer for Igfbp7
tgccctccatgaaataccac

SEQ ID NO: 50 - DNA, 20 bp, Artificial
  primer for Igfbp7
ggctgtctgagagcaccttt

SEQ ID NO: 51 - DNA, 20 bp, Artificial
  primer for CIP
tagctactcggcccaagtct

SEQ ID NO: 52 - DNA, 20 bp, Artificial
  primer for CIP
atcccatgaggaatttcagg

SEQ ID NO: 53 - DNA, 20 bp, Artificial
  primer for PRKG1
tccaacattccagagccttc

SEQ ID NO: 54 - DNA, 23 bp, Artificial
  primer for PRKG1
ttttcatagtgggtctcttcgag

SEQ ID NO: 55 - DNA, 20 bp, Artificial
  primer for Beta-actin
gatctggcaccacaccttct

SEQ ID NO: 56 - DNA, 20 bp, Artificial
  primer for Beta-actin
ggggtgttgaaggtctcaaa

SEQ ID NO: 57 - DNA, 21 bp, Artificial
  GATA4 binding motif
catttatcattatctagggca

SEQ ID NO: 58 - DNA, 17 bp, Artificial
  GATA4 binding motif
agaaattatcgccaggg

SEQ ID NO: 59 - DNA, 19 bp, Artificial
  GATA4 binding motif
aatgcccggcattatctaa

SEQ ID NO: 60 - DNA, 21 bp, Artificial
  GATA4 binding motif
catttatcattatctagggcc

SEQ ID NO: 61 - DNA, 17 bp, Artificial
  GATA4 binding motif
agaaattatcgccaggg

SEQ ID NO: 62 - DNA, 19 bp, Artificial
  GATA4 binding motif
aatgtcctgcaatatctaa

SEQ ID NO: 63 - DNA, 21 bp, Artificial
  GATA4 binding motif
catttatcattatctagggca

SEQ ID NO: 64 - DNA, 17 bp, Artificial
  GATA4 binding motif
agaaattatcgccaggg

SEQ ID NO: 65 - DNA, 19 bp, Artificial
  GATA4 binding motif
aatgtcctgcaatatctaa

SEQ ID NO: 66 - DNA, 21 bp, Artificial
  GATA4 binding motif
catttatcattacctagggca

SEQ ID NO: 67 - DNA, 17 bp, Artificial
  GATA4 binding motif
agaaattatcgccaggg

SEQ ID NO: 68 - DNA, 19 bp, Artificial
  GATA4 binding motif
aatgtcctacagtatctaa